SEQUENCE LISTING (36 sequences)

SEQ ID NO: 1 - protein, 213 aa, Rhodobacter sphaeroides
MTIRHHVSDALLTAYAAGTLSEAFSLVVATHLSLCDECRARAGALDAVGGSLMEETAPVALSEGSLASVMAQLDRQIQRPAPARRADPRAPAPLADYVGRRLEDVRWRTLGGGVRQAILPTGGEAIARLLWIPGGQAVPDHGHRGLELTLVLQGAFRDETDRFGAGDIEIADQELEHTPVAERGLDCICLAATDAPLRFNSFLPKLVQPFFRI

SEQ ID NO: 2 - protein, 181 aa, Rhodobacter sphaeroides
MTDKSDRTDWVALMRAIRDHRDEAAFAELFQHFAPKVKGFLMKSGSVASQAEECAQDVMATVWQKAHLFDPSRASVATWIFTIARNRRIDGLRKDRQPEPEDLFWGPDSEPDQADVYEMQQENARLGRAIARLPEAQRALIERAFFGDLTHRELAAETGLPLGTIKSRIRLALDRLRQHMS

SEQ ID NO: 3 - DNA, 30 bp, Artificial Sequence
  Synthetic oligonucleotide
tgatccnnnnnnnnnnnnnnnnnntagtga

SEQ ID NO: 4 - DNA, 30 bp, Artificial Sequence
  Synthetic oligonucleotide
tgatccnnnnnnnnnnnnnnnnnntagtaa

SEQ ID NO: 5 - DNA, 30 bp, Artificial Sequence
  Synthetic oligonucleotide
tgatccnnnnnnnnnnnnnnnnnntatctg

SEQ ID NO: 6 - DNA, 30 bp, Artificial Sequence
  Synthetic oligonucleotide
tgatccnnnnnnnnnnnnnnnnnntaagaa

SEQ ID NO: 7 - DNA, 31 bp, Artificial Sequence
  Synthetic oligonucleotide
tcatccnnnnnnnnnnnnnnnnnnntagcct

SEQ ID NO: 8 - DNA, 30 bp, Artificial Sequence
  Synthetic oligonucleotide
tgatccnnnnnnnnnnnnnnnnnntaagag

SEQ ID NO: 9 - DNA, 13 bp, Artificial sequence
  Synthetic oligonucleotide
ccttgggcgacag

SEQ ID NO: 10 - DNA, 13 bp, Artificial Sequence
  Synthetic oligonucleotide
gacatgtgttttt

SEQ ID NO: 11 - DNA, 13 bp, Artificial Sequence
  Synthetic oligonucleotide
ggaagcgggcccg

SEQ ID NO: 12 - DNA, 13 bp, Artificial Sequence
  Synthetic oligonucleotide
gactggcccggcc

SEQ ID NO: 13 - DNA, 14 bp, Artificial Sequence
  Synthetic oligonucleotide
ccggagccgccttc

SEQ ID NO: 14 - DNA, 13 bp, Artificial Sequence
  Synthetic oligonucleotide
aaccgtcgcttaa

SEQ ID NO: 15 - DNA, 14 bp, Artificial Sequence
  Synthetic oligonucleotide
caagtgagacccga

SEQ ID NO: 16 - DNA, 13 bp, Artificial Sequence
  Synthetic oligonucleotide
gaggtcgggcctc

SEQ ID NO: 17 - DNA, 13 bp, Artificial Sequence
  Synthetic oligonucleotide
gaacgcgcggccc

SEQ ID NO: 18 - DNA, 13 bp, Artificial Sequence
  Synthetic oligonucleotide
aaactaaagcttt

SEQ ID NO: 19 - DNA, 13 bp, Artificial Sequence
  Synthetic oligonucleotide
catggggatatct

SEQ ID NO: 20 - DNA, 13 bp, Artificial Sequence
  Synthetic oligonucleotide
cccgcacggggtc

SEQ ID NO: 21 - DNA, 13 bp, Artificial Sequence
  Synthetic oligonucleotide
acgtgaacaagat

SEQ ID NO: 22 - DNA, 13 bp, Artificial Sequence
  Synthetic oligonucleotide
gacctgatccggc

SEQ ID NO: 23 - DNA, 13 bp, Artificial Sequence
  Synthetic oligonucleotide
gctcgccgccatc

SEQ ID NO: 24 - DNA, 13 bp, Artificial Sequence
  Synthetic oligonucleotide
gggtgcgcgaccc

SEQ ID NO: 25 - DNA, 12 bp, Artificial Sequence
  Synthetic oligonucleotide
gcgccgattgca

SEQ ID NO: 26 - DNA, 13 bp, Artificial Sequence
  Synthetic oligonucleotide
gctgatcgtcggc

SEQ ID NO: 27 - DNA, 13 bp, Synthetic oligonucleotide
gagcggtctcgcg

SEQ ID NO: 28 - DNA, 13 bp, Artificial Sequence
  Synthetic oligonucleotide
gcaccggctggcc

SEQ ID NO: 29 - DNA, 13 bp, Artificial Sequence
  Synthetic oligonucleotide
gttacctccttgc

SEQ ID NO: 30 - DNA, 13 bp, Artificial Sequence
  Synthetic oligonucleotide
ccttccatcatct

SEQ ID NO: 31 - DNA, 11 bp, Artificial Sequence
  Synthetic oligonucleotide
ctggcccgcag

SEQ ID NO: 32 - DNA, 13 bp, Artificial Sequence
  Synthetic oligonucleotide
gatggtcttcagc

SEQ ID NO: 33 - DNA, 13 bp, Artificial Sequence
  Synthetic oligonucleotide
gatgcgactctcc

SEQ ID NO: 34 - DNA, 14 bp, Artificial Sequence
  Synthetic oligonucleotide
ccatggaaaacgag

SEQ ID NO: 35 - DNA, 14 bp, Artificial Sequence
  Synthetic oligonucleotide
ccgggcggcggcat

SEQ ID NO: 36 - DNA, 13 bp, Artificial Sequence
  Synthetic oligonucleotide
gaaggaatgttca

(19) United States
(12) Patent Application Publication
(10) Pub. No.: US 2010/0299781 A1
(43) Pub. Date: Nov. 25, 2010

(54) RESPONSES TO SINGLET OXYGEN

(76) Inventors: Timothy J. Donohue, Middleton, WI (US); Jennifer Rachel Anthony, San Lorenzo, CA (US); Kristin L. Warczak, Franklin, WI (US); Yann DuFour, Madison, WI (US); Heather Green, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 12/410,066

(22) Filed: Mar. 24, 2009

Related U.S. Application Data
(63) Continuation-in-part of application No. 11/410,431, filed on Apr. 25, 2006.
(60) Provisional application No. 60/674,470, filed on Apr. 25, 2005.

(51) Int. Cl. C12N 15/74; C12N 1/20; C12N 1/12; C12N 15/82

(52) U.S. Cl. 800/278; 435/471; 435/252.1; 435/257.1

(57) ABSTRACT

The physiological response of an organism to singlet oxygen is altered by modulating the interaction between an anti-sigma factor, ChrR, and a sigma factor, σ^{E}, or by altering expression of a gene product required for viability in the presence of singlet oxygen.

### CROSS-REFERENCE TO RELATED APPLICATIONS

This application is a continuation-in-part of U.S. patent application Ser. No. 11/410,431, filed Apr. 25, 2006, which claimed the benefit of U.S. Provisional Patent Application Ser. No. 60/674,470, filed Apr. 25, 2005. Each application is incorporated herein by reference as if set forth in its entirety.

### STATEMENT REGARDING FEDERALLY SPONSORED RESEARCH OR DEVELOPMENT

This invention was made with United States government support awarded by the following agencies: National Institute of General Medical Science, GM37509 & GM075273; and Department of Energy, DE-FG02-05ER15653 & ER63232-1018220-0007203. The United States has certain rights in this invention.

### BACKGROUND

The invention relates generally to modulating physiological responses to singlet oxygen in bacterial cells, algae or plants. Singlet oxygen (¹O₂) is produced by enzymes such as peroxidases and oxidases or as byproduct of various processes such as photosynthesis. Kochevar I, “Singlet oxygen signaling: from intimate to global,” STKE 204:pe7 (2004). In the photosynthetic process, input light energy converts water (H₂O) and carbon dioxide (CO₂) to oxygen (O₂) and sugar. Cellular respiration subsequently converts some of the sugar into chemical energy in the form of ATP. The conversion is associated with chlorophyll, a green pigment common to all photosynthetic cells. Although O₂ is a relatively non-reactive chemical, when exposed to high-energy or electron-transferring chemical reactions, it can be converted to highly reactive chemical forms collectively designated as “reactive oxygen species” (ROS). ROS are generally considered toxic to organisms because they oxidize carbohydrates, DNA, lipids and proteins, breaking down normal cellular, membrane and reproductive functions. Ultimately, at toxic ROS levels, a chain reaction of cellular oxidation can result in disease or lethality.

In phototrophs, including plants, light energy excites chlorophyll pigments in the light harvesting complexes to a triplet state. At some frequency, an energy transfer from the excited triple state chlorophyll pigments to ground-state O₂ generates ¹O₂ which, as a strong oxidant, can destroy membrane integrity, abolish biomolecular function, and reduce photochemical activity by inactivating photosynthetic enzymes.

Because excited triplet-state chlorophyll pigments and ground-state oxygen are found in close proximity to one another, many phototrophs exhibit some natural defenses against ¹O₂. For example, carotenoids, fat-soluble, anti-oxidant pigments found within the photosynthetic apparatus, quench ¹O₂. Telfer A, “What is β-carotene doing in the photosystem II reaction centre,” Phil. Tans. R. Soc. Lond. 357:1431-1440 (2002). Carotenoids include, but are not limited to, β-carotene, zeaxanthin and tocopherols. If not completely quenched by carotenoids or other suitable compounds, ¹O₂ can specifically trigger upregulation of genes that encode proteins involved in the molecular defense against photo-oxidative stress. For example, a network of upregulated plant genes maintains a balance between ROS-scavenging proteins and ROS-producing proteins. Mittler R, “Reactive oxygen gene network of plants,” TRENDS in Plant Sci. 9:490-498 (2004). In bacteria, a set of sigma factors, interchangeable RNA polymerase subunits responsible for recognizing transcriptional promoters, maintain essential housekeeping functions and facilitate host response to specific environmental stresses, including ROS. A constitutively-expressed, principal sigma factor is responsible for transcribing essential housekeeping genes. Other sigma factors, transcriptionally- or post-translationally-activated in response to stresses, recognize promoters upstream of genes involved in the response to stresses. Sigma factors are themselves regulated by anti-sigma factors that bind to a specific sigma factor and inhibit that sigma factor's ability to recognize a promoter.

Activation of sigma factors has been studied, inter alia, in Rhodobacter sphaeroides, a member of the α-subdivision of Proteobacteria and a facultative phototroph. R. sphaeroides is among the most metabolically diverse organisms known, being capable of growth under a wide variety of growth conditions. In addition to being photosynthetic, R. sphaeroides possesses additional energy-acquiring mechanisms including lithotrophy, aerobic respiration and anaerobic respiration. SigmaE (σ^{E}), a 19.2 kDa alternative sigma factor encoded by rpoE and related to members of the extra-cytoplasmic function (ECF) subfamily of eubacterial RNA polymerase sigma factors, is increased following environmental stress in R. sphaeroides. σ^{E} directs transcription from rpoE P1, a promoter for the rpoEchrR operon, and from cycA P3, a promoter for cytochrome c₂. Newman J, et al, “The Rhodobacter sphaeroides ECF sigma factor, σ^{E}, and the target promoters cycA P3 and rpoE P1,” J. Mol. Biol. 294:307-320 (1999), incorporated herein by reference as if set forth in its entirety. Basal σ^{E} activity, however, is quite low because it is complexed with a zinc-dependent anti-sigma factor, ChrR. ChrR loses its ability to inhibit σ^{E} if zinc is removed, or if a zinc-binding domain of the N-terminal domain is removed. Newman J, et al., “The importance of zinc-binding to the function of Rhodobacter sphaeroides ChrR as an anti-sigma factor,” J. Mol. Biol. 313:485-499 (2001), incorporated herein by reference as if set forth in its entirety.

GenBank Accession No. AAB17905 (SEQ ID NO:1), discloses the full-length R. sphaeroides ChrR sequence. ChrR with a C38R mutation prevented binding to σ^{E}. See Newman et al. (1999), supra. Likewise, ChrR with a C35S or a C38S mutation prevented binding to σ^{E}. See Newman et al. (2001), supra. Furthermore, a ChrR with a C187/189S mutation was shown to prevent binding to σ^{E}. Id. In addition, ChrR with a H6A mutation, a H31A mutation, a C35A mutation or a C38A mutation cannot bind zinc and ultimately cannot bind σ^{E}.

GenBank Accession No. AAB17906 (SEQ ID NO:2) discloses the full-length R. sphaeroides σ^{E} sequence. Mutations in region 2.1 (amino acids 22 to 46 of SEQ ID NO:2) of σ^{E} alter the interaction between ChrR and σ^{E}. Anthony J, et al., “Interactions between the Rhodobacter sphaeroides ECF sigma factor, σ^{E}, and its anti-sigma factor, ChrR,” J. Mol. Biol. 341:345-360 (2004), incorporated herein by reference as if set forth in its entirety. In particular, σ^{E} with a K38E mutation, a K38R mutation or a M42A mutation were less sensitive to ChrR both in vivo and in vitro.

Because ¹O₂ affects many organisms (including, but not limited to, bacteria, plants, animals and humans), the components of the biological response to ¹O₂ find application in medicine, agriculture, biotechnology and bioenergy production systems. Animals and plants use ¹O₂ to defend against microbial pathogens. Davies M, “Reactive species formed on proteins exposed to singlet oxygen,” Photochem. Photobiol. Sci. 3:17-25 (2004), incorporated herein by reference as if set forth in its entirety. For the foregoing reasons, there is a desire to manipulate physiological responses to ¹O₂ in animals, bacteria and plants. There are many advantages of studying responses to ¹O₂ in R. sphaeroides. First, one can control the formation of significant amounts of ¹O₂. Also, biochemical and genetic systems are available to study the response to ¹O₂ in vivo and in vitro, including an Affymetrix gene chip (Affymetrix; Santa Clara, Calif.), LC/MS proteomics and computation approaches.

### BRIEF SUMMARY

The present invention relates to observations by the inventors relating to genes required for viability of R. sphaeroides in the presence of ¹O₂ which can be generated during photosynthesis. Specifically, changes in the interaction between alternative sigma factor σ^{E} and its anti-sigma factor ChrR affects expression of genes required for viability of R. sphaeroides in the presence of ¹O₂. Although homologs of σ^{E} and ChrR have been identified computationally in other bacteria, their involvement in a cellular response to ¹O₂ has not heretofore been noted.

As the inventors detail below, ¹O₂ typically has detrimental effects upon cells, but cells can avoid or overcome the effects by increasing σ^{E}, which is ordinarily complexed with ChrR. In the presence of ¹O₂, ChrR and σ^{E} dissociate and synthesis of σ^{E} increases, allowing free σ^{E} to bind to a core RNA polymerase, facilitating transcription of a regulon involved in attenuating physiological effects of ¹O₂. This observation suggests that σ^{E} or ChrR can be manipulated to exploit the response of cells and organisms to ¹O₂. Even though some of the observations were made in R. sphaeroides, the invention is not intended to be limited to this single prokaryote, as responses to ¹O₂ are present in many other species, including both photosynthetic and non-photosynthetic prokaryotes and eukaryotes.

The observation can be exploited to inhibit or prevent microbial survival, by preventing dissociation between ChrR and σ^{E} or by reducing the extent of dissociation in the microbes. In the presence of ¹O₂, the microbes would succumb to damage caused by increased oxidative stress.

The observation can alternatively be exploited to increase efficiency of microbially-catalyzed commercial processes for generating commodity chemicals such as, but not limited to, acetic acid and other organic acids, acetone, acrylamide, butanol, ethanol, glycerol, isoprenoids, quinines, and pigments as well as strategic chemicals with potential use as lubricants or biofuels since some of the gene products involved in this conserved response modify hydrocarbons within the cell membrane. Nagasawa T & Yamada H, “Microbial production of commodity chemicals,” 67 Pure & Appl. Chem. 1241-1256 (1995), incorporated herein by reference as if set forth in its entirety. In particular, photosynthetic organisms for use in such processes can be engineered to inhibit or eliminate binding between ChrR and σ^{E}, such that when the microbe finds itself in the presence of ¹O₂, it readily overcomes any toxic effects by mobilizing its increased available supply of σ^{E} to initiate transcription of the protective regulon, ensuring robust production from the process, notwithstanding the presence of ¹O₂. Increasing production of these or other commodity chemicals involves inhibiting the interaction between ChrR and σ^{E} in the presence of ¹O₂ so that the microbe continues to produce a desired commodity chemical notwithstanding oxidative stress. In one approach, the microbe can be engineered either to contain a mutated ChrR that cannot bind σ^{E}, or to lack ChrR entirely. A similar effect can be obtained by engineering a microbe for use in the process where the microbe contains a mutated σ^{E} relative to wild-type σ^{E} such that the mutated sigma factor cannot bind ChrR, or binds ChrR only weakly. In some embodiments, the photosynthetic organism is a bacterium, an alga or a plant. In some embodiments, the photosynthetic organism is R. sphaeroides. In some embodiments, σ^{E} is modified relative to wild type by engineering a K38E mutation, a K38R mutation or a M42A mutation in σ^{E}. In some embodiments, ChrR is modified relative to wild type by engineering a C35S mutation, a C38S mutation, a C38R mutation or a C187/189S in ChrR.

In another aspect, the observation can be exploited by protecting the phototroph from toxic effects of ¹O₂ by looking beyond the direct interaction of σ^{E} and ChrR, to the genes transcribed directly by σ^{E} or genes whose expression is increased by a σ^{E}-dependent transcription factor. For example, where the available supply of σ^{E} is not, or cannot, be increased as noted above, the phototroph, especially a plant, can be engineered to increase expression of genes that encode protective proteins. For example, CfaS, shown herein to be upregulated by σ^{E}, encodes cyclopropane-fatty-acyl-phospholipid synthase which catalyzes the generation of cyclopropane fatty acids by adding a methylene bridge across a double bond of a fatty acid. In lipid bilayers, ¹O₂ can hydroxylate unsaturated fatty acids and membrane-destabilizing lipid peroxides can form. On the other hand, ¹O₂ cannot hydroxylate cyclopropane fatty acids in the bilayers, so lipid peroxides cannot form and the phototrophs are protected from oxidative stress. Conversely, unwanted or invasive plant species can be made more susceptible to oxidative stress by engineering the phototroph to downregulate genes that encode protective proteins, such as CfaS, which is known to modify the fatty acid composition of the membrane and result in accumulation of hydrocarbons of strategic value as biofuels, lubricants, etc.

In another aspect, the present invention is summarized in that a consensus promoter responsive to σ^{E} is disclosed as SEQ ID NO:3. In some embodiments, the nucleic acid residues at positions 2, 6, 12, 16, 17 and 20 are G; at position 11 is A; and at positions 19, 22 and 26 are C. The isolated nucleic acid sequence of SEQ ID NO:3 can be operably linked to a heterologous reporter gene or gene of interest to produce a genetic construct suitable for transfer into cells of a phototroph. Expression of the operably linked gene can thereby be placed under the control of σ^{E}. In so doing, not only can protective proteins be produced in the presence of ¹O₂, but any other protein, polypeptide, peptide or oligonucleotide of interest can be induced under such conditions. Similarly, a reporter gene can be provided so that the presence of ¹O₂ can be detected, observed and monitored.

It is an advantage of the present invention that it provides the skilled person with the tools for efficiently producing products of this response in phototrophic or non-phototrophic organisms while avoiding longstanding issues arising from the presence of ¹O₂ or allowing skilled persons to express gene products in microbes to produce such commodity or strategic chemicals either in the presence or absence of this ROS.

A second advantage is that the methods and compositions are non-toxic to the environment.

These and other features, aspects and advantages of the present invention will become better understood from the description that follows as well as from Dufour et al., J. Mol. Biol. 383:713-730 (2008), incorporated herein by reference as if set forth in its entirety. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

Abbreviations used in the drawings: PS—photosynthetically grown cells, Aero—cells grown by aerobic respiration, WT—wild type, ΔChrR—cells lacking the anti-sigma factor ChrR, Δσ^{E}—cells lacking both σ^{E} and ChrR.

FIG. 1 shows that the conditions that generate ¹O₂ increase R. sphaeroides' σ^{E} activity. Cells were grown in either steady-state cultures or were shifted from photosynthetic to aerobic conditions in the presence of light, which was either white, unfiltered light (light) or filtered light at >830 nm. The arrow indicates the time of shift. β-galactosidase activity from a σ^{E}-dependent reporter gene is shown.

FIG. 2 shows that cells require continued exposure to ¹O₂ to maintain increased σ^{E} activity. β-galactosidase activity from a σ^{E}-dependent reporter gene when photosynthetically grown cells are shifted to aerobic conditions in the presence or absence of light. Arrows indicate each shift.

FIG. 3 shows that ¹O₂ is bacteriocidal to a Δσ^{E} mutant when carotenoids are low. Aerobically grown wild type or Δσ^{E} cells were treated with methylene blue in the presence of light. The arrow indicates when methylene blue and light were added. (A) Optical density measurements (OD₅₀₀ₙₘ) and (B) viable plate counts (cfu/ml).

FIG. 4 identifies additional σ^{E}-dependent promoters. (A) Products of in vitro transcription reactions using reconstituted R. sphaeroides (Eσ^{E}) and the indicated potential promoter. As an additional control to demonstrate the σ^{E}-dependence of these transcripts, ChrR was added to indicated reactions. Note that the first four lanes were exposed to a phosphoscreen twice as long as the remainder of the gel to detect low abundance transcripts from the cycA P3 and Rsp1409 promoters. Experiments were repeated at least three times, with a representative gel shown. The σ^{E}-dependent transcripts appear as two products due to termination at different bases within the SpoT 40 transcriptional terminator on the template used. (B) Activity of selected σ^{E}-dependent promoters in R. sphaeroides. Shown are β-galactosidase levels (in Miller units) from the indicated promoter fused to lacZ in wild type cells (▪), ΔChrR cells (▪), or both Δσ^{E} and ΔChrR cells (□). All assays were performed in triplicate, with bars denoting the standard deviation from the mean.

FIG. 5 illustrate the consensus sequence of the σ^{E}-dependent promoter motif in R. sphaeroides (A) and other bacteria (B). FIG. 5A. shows the −35 and −10 sequence logos, which were obtained by sequence alignment of six σ^{E}-dependent promoters from R. sphaeroides. The two conserved −35 and −10 regions are separated by a spacer of 13-14 bp. The information content (I_{seq}) of each motif is indicated. FIG. 5B shows −35 and −10 motifs that were found upstream of the rpoE gene in 57 of the 73 selected microbial genomes. In both panels, the logos were produced with WebLogo, available from the web page of the software's creators, available on the internet at a site hosted by the University of California—Berkeley.

FIG. 6 illustrates the activity of σ^{E}-dependent promoters in R. sphaeroides. Promoter activity is represented by β-galatosidase activity (Miller units) in wild-type (gray), ΔchrR (black), and ΔrpoEchrR (white) cells. All assays were performed in triplicate, with bars representing standard deviations.

FIG. 7 shows three representative genomic regions enriched by the immunoprecipitation of DNA fragments using anti-σ^{E} antibodies (σ^{E}) or anti-β′ antibodies (β′) and identified by ChIP-chip. The data represent the log₂ of the ratio of the immunoprecipitated sample to the control sample as a function of probe location along the R. sphaeroides genome (coordinates are indicated in base pairs). Regions significantly enriched by anti-σ^{E} immunoprecipitation (p≦0.01) are indicated by Peak blocks. The locations of the annotated open reading frames are indicated by ORFs blocks (read forward or reverse if above or below the baseline, respectively). The positions and orientations of the known or predicted σ^{E} promoter are indicated by the arrow. The data were plotted using SignalMap™ v1.9 (NimbleGen Systems).

FIG. 8 illustrates the phylogeny of the σ^{E}-ChrR pair. Phylogenetic trees were constructed with the concatenated amino acid sequence of σ^{E} and ChrR (a) or RuvB, RpoD, and GyrB (b) using Bayesian inference. Numbers on branches indicate Bayesian posterior probabilities. Branches of the tree for closely related species were collapsed for clarity. In FIG. 8A proteobacteria classes are γ-proteobacteria (Shewanella sp. through Oceanobacter sp.), α-proteobacteria (Hyphomonas neptunium throughXanthobacter autotrophicus and Rhodopseudomonas palustris through Roseobacter sp.), beta proteobacterium (Acidovorax avenae citrulli), and delta proteobacterium (Myxococcus xanthus). In FIG. 8B proteobacteria classes are indicated on the right.

FIG. 9 illustrates the amino acid sequence alignment of σ^{E} homologs from a set of representative species containing σ^{E}-ChrR proteins. The histogram below the alignment represents the relative conservation score for each position based on alignment of all 73 σ^{E} homologs considered in this study. Arrows indicate residues predicted to contact DNA in the α35 region of the promoter based on alignment with E. coli σ^{E}. Sequences are identified by their locus ID.

### DETAILED DESCRIPTION OF PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

As used herein, a “phototroph” or “photosynthetic organism” refers to any organism that is capable of photosynthesis.

### EXAMPLES

### Example 1

### Role of σ^{E} in Response to ¹O₂

Methods

Bacterial strains and plasmids: R. sphaeroides 2.4.1 (wild-type, WT), R. sphaeroides with a mutant ChrR (ΔChrR) (chrR-1::drf, see Newman et al., supra.) or R. sphaeroides with both a mutant σ^{E} (Δσ^{E}) and ΔChrR (TF18; rpoEchrR-1::drf, see Schilke B & Donohue T, “ChrR positively regulates transcription of the Rhodobacter sphaeroides cytochrome c₂ gene,” J. Bacteriol. 177:1929-1937 (1995), incorporated herein by reference as if set forth in its entirety) were grown at 30° C. in Sistrom's succinate-based minimal medium A. Media used for growth of strains containing low-copy lacZ reporter plasmids was supplemented with 25 μg/ml kanamycin.

Growth conditions: For aerobic respiratory growth, 500 ml of media was bubbled with a mixture of 69% N₂, 30% O₂ and 1% CO₂ in the dark. Conversely, for photosynthetic growth, 500 ml of media was bubbled with a mixture of 95% N₂ and 5% CO₂ in front of an incandescent light source (10 W/m² as measured with a Yellow-Springs-Kettering model 6.5 A radiometer through a Corning 7-69, 620 to 110 nm filter).

To test the effects of ¹O₂, photosynthetic cultures were exposed to aerobic growth conditions (69% N₂, 30% O₂ and 1% CO₂) in the presence or in the absence of light (10 W/m²). Where indicated, light was passed through a 1283 filter (Kopp Glass; Pittsburgh, Pa.) that impedes >99% of light at wavelengths <770 nm, but transmits >45% of light at 830 nm and >80% of light at 900 nm. When using methylene blue (Sigma-Aldrich; St. Louis, Mo.) to generate ¹O₂, a final concentration of 1 μM was added to aerobic cultures in the presence or in the absence of incandescent light (10 W/m²). To test the effects of other ROS, 0.5 mM H₂O₂, 1 mM diamide or 1 mM paraquat (Sigma-Aldrich) was added to aerobic cultures.

All experiments were initiated when cultures reached ˜2×10⁸ cfu/ml to minimize light or O₂ limitation to photosynthetic and aerobic cells, respectively. To measure cell viability, samples were removed, diluted and plated in media supplemented with 25 μg/ml kanamycin to select for the rpoE P1::lacZ reporter plasmid. The whole cell abundance of carotenoids was measured as described in Cohen-Bazire G, et al., “Kinetic studies of pigment synthesis by non-sulfur purple bacteria,” J. Cell Physiol. 49:25-68 (1957).

Determining promoter activity: In vivo promoter activity was determined by measuring β-galactosidase activity from a low copy rpoE P1::lacZ reporter plasmid or a trxA::lacZ reporter plasmid. The promoter for the thioredoxin gene (trxA, −214 to +27 relative to the known transcription initiation site) was fused to lacZ and mobilized into R. sphaeroides.

β-galactosidase activity (units/ml of culture) was calculated as follows: (A₄₂₀×1000)/(Cell volume in assay (ml)×Time of assay (min)). Culture density was typically monitored by measuring A₆₀₀ in a BioSpec 1601 spectrophotometer (Schimatzu; Columbia, Md.). The density of cultures treated with methylene blue was monitored at 500 nm because methylene blue absorbs light between 609-668 nm. The differential rate of β-galactosidase synthesis was determined by calculating the slope from plots of enzyme activity (units/ml of culture) against optical density. All experiments were repeated a minimum of three times with differential rates of β-galactosidase synthesis typically deviating less than two-fold between experiments.

In vitro promoter activity was determined by using plasmid templates in which a test promoter was cloned into a plasmid (pRKK96) containing a known transcriptional terminator. Standard assay conditions mixed recombinant His₆-σ^{E} (50 nM) with R. sphaeroides core RNA polymerase (50 nM) for 30 minutes at 30° C. in transcription buffer (40 mM Tris-HCl pH 7.9, 200 mM KCl, 10 mM Mg acetate, 1 mM DTT, and 62.5 mg/ml acetylated bovine serum albumin). Next, 20 nM of plasmid DNA was added and incubated for 30 minutes before transcription was initiated by adding nucleotide triphosphates. Newman et al., supra. Samples were incubated for 20 minutes before RNA products were analyzed by 6% (wt/vol) denaturing polyacrylamide gel electrophoresis, and products were visualized on a phosphorimager (Molecular Dynamics, Sunnyvale, Calif.

Identification of σ^{E} target genes: Triplicate cultures of WT and ΔChrR were grown aerobically to ˜2−3×10⁸ CFU/ml. RNA was isolated and cDNA was synthesized, labeled and hybridized to R. sphaeroides GeneChip Custom Express microarrays (Affymetrix). After data extraction using Affymetrix MAS 5.0 software, data sets were imported into GeneSpring software (Silicon Genetics; Redwood City, Calif.) for normalization and analysis (Gene Expression Omnibus (GEO) accession number GSE2219).

Candidate σ^{E} promoters (extending ˜200 bp upstream of the predicted start of translation, Table 1) were amplified from 20 ng of WT chromosomal DNA in EasyStart PCR tubes (Molecular BioProducts; San Diego, Calif.) with 2.5 units Pfu Turbo (Stratagene; La Jolla, Calif.). PCR products were cloned into pRKK96 for in vitro assays or into a lacZ reporter plasmid (pRKK200) for determining activity in vivo.

TABLE 1
Genes with RNA expression levels ≧3-fold in ΔChrR strain.^{1,2,3}

| | | | Fold | Common | |
|---|---|---|---|---|---|
| ORF | ΔChrR¹ | WT | Increase² | Name | Description³ |
| RSP0028 | 0.438 | 0.111 | 3.9 | | Putative short-chain |
| | | | | | dehydrogenase/reductase |
| RSP0103 | 1.451 | 0.376 | 3.9 | nuoE | NADH dehydrogenase (ubiquinone), |
| | | | | | 24-kDa subunit |
| RSP0105 | 1.525 | 0.131 | 11.7 | nuoG | Respiratory-chain NADH |
| | | | | | dehydrogenase 75-kDa subunit |
| RSP0107 | 2.148 | 0.523 | 4.1 | nuoI | 7Fe ferredoxin:3Fe-4S ferredoxin:4Fe-4S |
| | | | | | ferredoxin, iron-sulfur-binding |
| | | | | | domain |
| RSP0136 | 0.378 | 0.103 | 3.7 | | Putative integrase for prophage CP- |
| | | | | | 933U |
| RSP0216 | 0.503 | 0.0602 | 8.4 | | Hypothetical |
| RSP0258 | 48.66 | 14.19 | 3.4 | pufA | LHI α, light-harvesting B875 protein |
| RSP0261 | 1.147 | 0.287 | 4.0 | bchY | Chlorophyllide reductase, BchY |
| | | | | | subunit |
| RSP0262 | 0.607 | 0.0783 | 7.7 | bchX | Chlorophyllide reductase, BchX |
| | | | | | subunit |
| RSP0286 | 2.301 | 0.489 | 4.7 | bchB | Light-independent protochlorophyllide |
| | | | | | reductase |
| RSP0287 | 1.068 | 0.203 | 5.3 | bchH | Magnesium-chelatase subunit H |
| RSP0288 | 3.402 | 1.012 | 3.4 | bchL | Light-independent protochlorophyllide |
| | | | | | reductase iron protein |
| RSP0300 | 0.328 | 0.106 | 3.1 | | ABC branched chain amino acid |
| | | | | | transporter, inner membrane subunit |
| RSP0351 | 2.035 | 0.0421 | 48.3 | | Pseudogene of D-threo-aldose 1- |
| | | | | | dehydrogenase |
| RSP0464 | 0.348 | 0.0957 | 3.6 | | Putative protease |
| RSP0473 | 0.47 | 0.149 | 3.2 | | Phospholipase-D family protein |
| RSP0483 | 0.483 | 0.16 | 3.0 | | |
| RSP0601 | 20.71 | 0.541 | 38.3 | rpoH2 | RNA polymerase σ factor RpoH2 (σ- |
| | | | | | 32 group, heat shock) |
| RSP0770 | 0.173 | 0.0524 | 3.3 | | |
| RSP0799 | 7.747 | 2.212 | 3.5 | | Conserved hypothetical protein |
| RSP0820 | 0.817 | 0.201 | 4.1 | | Putative cytochrome B561 |
| RSP0947 | 0.432 | 0.129 | 3.4 | | Hypothetical protein |
| RSP1008 | 0.501 | 0.132 | 3.8 | | |
| RSP1025 | 4.55 | 1.287 | 3.5 | | Conserved hypothetical protein |
| RSP1026 | 2.13 | 0.65 | 3.3 | | |
| RSP1087 | 8.799 | 1.865 | 4.7 | | Short-chain dehydrogenase/reductase |
| | | | | | family member |
| RSP1088 | 7.219 | 0.338 | 21.4 | | Hypothetical protein |
| RSP1089 | 4.204 | 0.573 | 7.3 | | Sugar/cation symporter, GPH family |
| RSP1090 | 5.57 | 0.0311 | 179.3 | | Putative cyclopropane/cyclopropene |
| | | | | | fatty acid synthesis protein |
| RSP1091 | 31.93 | 1.968 | 16.2 | | Putative cyclopropane/cyclopropene |
| | | | | | fatty acid synthesis protein, flavin |
| | | | | | amine oxidase |
| RSP1092 | 17.55 | 1.399 | 12.5 | rpoE | RNA polymerase σ factor RpoE (ECF |
| | | | | | group, extracytoplasmic function) |
| RSP1263 | 0.273 | 0.0749 | 3.6 | | |
| RSP1409 | 48.75 | 0.302 | 161.6 | | Beta-Ig-H3/fasciclin domain |
| RSP1410 | 2.606 | 0.709 | 3.7 | | Conserved hypothetical protein |
| RSP1504 | 0.481 | 0.056 | 8.6 | | Conserved hypothetical protein |
| RSP1540 | 0.973 | 0.297 | 3.3 | | Predicted secreted hydrolase |
| RSP1546 | 16.61 | 3.279 | 5.1 | bfr | Bacterioferritin |
| RSP1591 | 4.283 | 0.675 | 6.3 | | Predicted glutathione S-transferase, C- |
| | | | | | terminal |
| RSP1619 | 0.265 | 0.0271 | 9.8 | | Hypothetical |
| RSP1656 | 0.123 | 0.0269 | 4.6 | | Hypothetical |
| RSP1759 | 8.572 | 2.632 | 3.3 | | Hypothetical |
| RSP1760 | 6.211 | 1.166 | 5.3 | | Hypothetical protein |
| RSP1852 | 19.85 | 1.922 | 10.3 | | Conserved hypothetical protein |
| RSP1853 | 1.235 | 0.183 | 6.8 | TrkH2 | Potassium uptake transporter, |
| | | | | | transmembrane component, TrkH |
| RSP1895 | 1.454 | 0.145 | 10.1 | | Small-conductance mechanosensitive |
| | | | | | ion channel |
| RSP1924 | 0.341 | 0.1 | 3.4 | | Probable biotin synthase |
| RSP2030 | 0.294 | 0.0458 | 6.4 | | Putative sensor histidine kinase |
| | | | | | (fragment) |
| RSP2037 | 0.619 | 0.191 | 3.2 | | Conserved hypothetical protein |
| RSP2066 | 0.908 | 0.13 | 7.0 | | Hypothetical |
| RSP2143 | 5.775 | 1.315 | 4.4 | | DNA photolyase, cryptochrome 1 |
| | | | | | apoprotein (blue-light photoreceptor) |
| RSP2144 | 11.1 | 1.123 | 9.9 | cfaS | Cyclopropane-fatty-acyl-phospholipid |
| | | | | | synthase (CfaS) |
| RSP2145 | 5.831 | 1.483 | 3.9 | trgA | Tellurite resistance protein |
| RSP2235 | 0.393 | 0.0335 | 11.7 | | Conserved hypothetical protein |
| RSP2268 | 4.223 | 0.991 | 4.3 | | Metallo β lactamase superfamily |
| RSP2294 | 2.737 | 0.896 | 3.1 | gloB | Putative hydroxyacylglutathione |
| | | | | | hydrolase (glyoxalase II) (GLX II) |
| | | | | | protein hydroxyacylgluta |
| RSP2314 | 4.134 | 1.12 | 3.7 | | Oxidoreductase - Aldo/keto reductase |
| | | | | | family: chromogranin/secretogranin |
| RSP2315 | 3.96 | 1.056 | 3.8 | | Conserved hypothetical protein |
| RSP2381 | 0.258 | 0.0597 | 4.3 | | Putative 3-methyladenine DNA |
| | | | | | glycosylase |
| RSP2389 | 2.144 | 0.0744 | 28.8 | | Putative glutathione peroxidase |
| RSP2390 | 1.758 | 0.391 | 4.5 | acuC1 | Putative acetoin utilization protein |
| RSP2391 | 0.469 | 0.0957 | 4.9 | | Putative ABC transporter (permease) |

¹ΔChrR: R. sphaeroides WT with trimethoprim cartridge inserted into ChrR.
²Increase in RNA abundance from comparing transcript levels in WT and ΔChrR cells. Data has been deposited at GEO under accession number GSE2219.
³Function known or predicted by genome annotation.

Results

Conditions that generate ¹O₂ within the photosynthetic apparatus increase R. sphaeroides activity: Mutations that inactivate an early enzyme in carotenoid biosynthesis, CrtB, cause a small increase in σ^{E} activity (data not shown). Since carotenoids play a protective role against ¹O₂, it was determined whether ¹O₂ directly affected σ^{E} activity.

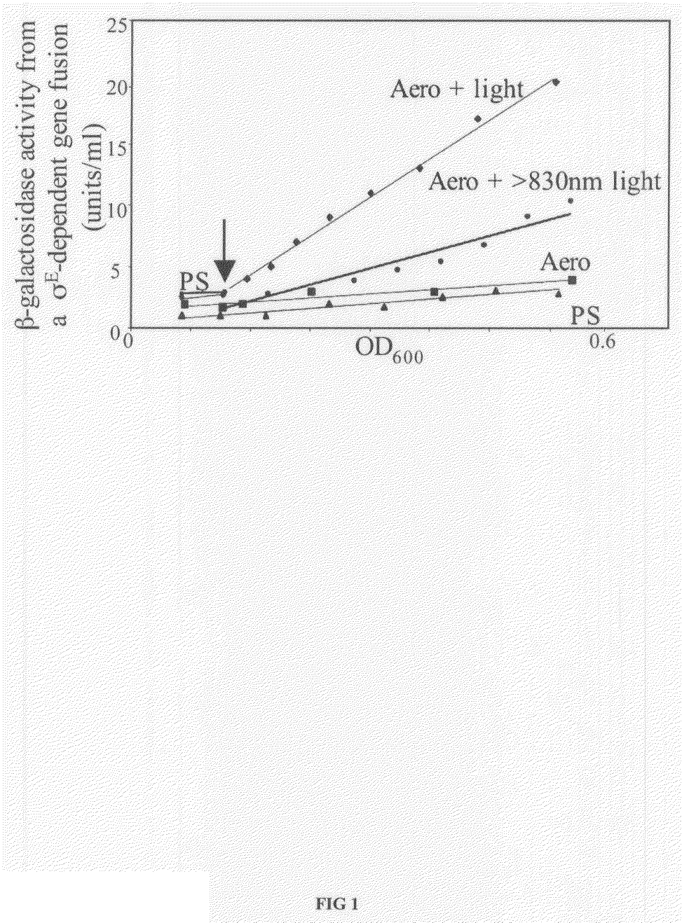

To determine if R. sphaeroides σ^{E} activity responds to ¹O₂, we examined the differential rate of β-galactosidase synthesis from a σ^{E}-dependent rpoE P1::lacZ reporter fusion after anaerobic, photosynthetic cells were exposed to O₂ in the presence of light. After exposure to O₂, the cells maintain approximately the same doubling rate, since O₂ is used as a respiratory electron acceptor. However, after exposure to O₂, the differential rate of β-galactosidase synthesis from the σ^{E}-dependent promoter increased ˜10-fold (from 6 to 65) when compared to a control culture grown under either a steady state photosynthetic condition (light in the absence of O₂) or a respiring condition (30% O₂) (FIG. 1 and Table 2).

TABLE 2
Differential rates of β-galactosidase synthesis from
the σ^{E}-dependent rpoE P1::lacZ reporter under conditions
that either do (+) or do not (−) generate ¹O₂.^{1,2}

| Strain | Growth | ¹O₂ | Rate |
|---|---|---|---|
| WT | PS | − | 6 |
| WT | Aero | − | 8 |
| WT | PS → Aero + light | + | 65 |
| WT | PS → Aero (dark) | − | 8 |
| WT | PS (>830 nm) | − | 2 |
| WT | PS → Aero (>830 nm) | + | 35 |

¹Aero = cells grown by aerobic respiration (30% O₂),
²PS = cells grown photosynthetically.

This transcriptional response was maintained throughout the experiment, suggesting that σ^{E} activity was sustained. There was less than a two-fold increase in the differential rate of β-galactosidase synthesis from the rpoE P1::lacZ reporter fusion when photosynthetic cells were shifted to aerobic conditions in the dark (Table 2). This was expected since little ¹O₂ is made under this condition due to lack of light needed to produce triplet state chlorophyll molecules. From these results, one can conclude that the combination of light and O₂, conditions known to generate ¹O₂ within the photosynthetic apparatus, are required for this transcriptional response.

Control experiments indicated that this response was dependent on σ^{E} since the differential rate of a β-galactosidase synthesis from the rpoE P1::lacZ reporter fusion in Δσ^{E} cells (<1 unit) did not increase upon exposure to ¹O₂. Δσ^{E} cells grow under these conditions, presumably because the carotenoids within the photosynthetic apparatus quench ¹O₂. In addition, it appears that ¹O₂ does not fully induce σ^{E} activity since the differential rate of β-galactosidase synthesis from the rpoE P1::lacZ reporter fusion in WT cells exposed to ¹O₂ was 10-fold less than that seen in ΔChrR cells (65 versus 650).

Wavelengths of light that excite chlorophyll pigments are sufficient to increase σ^{E} activity: If production of ¹O₂ by the photosynthetic apparatus was responsible for this transcriptional response, then wavelengths of light known to generate triplet state chlorophyll molecules within the light harvesting complexes should increase σ^{E} activity. R. sphaeroides contains two light harvesting complexes, B800-850 and B875, named for their absorption maxima in the near infrared. To determine if light absorbed by the light harvesting complexes could cause this response, we looked at the action spectrum of this transcriptional response. Under photosynthetic conditions with light that was filtered to remove wavelengths <830 nm, the differential rate of β-galactosidase synthesis from the σ^{E}-dependent promoter was an ˜4-fold lower than that observed with cells grown in white light (Table 2), presumably because the cells grow slower when light <830 nm is removed. However, there was an ˜17-fold increase in the differential rate of β-galactosidase synthesis when cultures illuminated with >830 nm light were exposed to O₂ (Table 2). The magnitude of this response was similar to that observed when photosynthetic cells were exposed to O₂ and white light (˜17-fold versus ˜10-fold, Table 2). Thus, wavelengths of light that excite the light harvesting complexes are sufficient to increase σ^{E} activity.

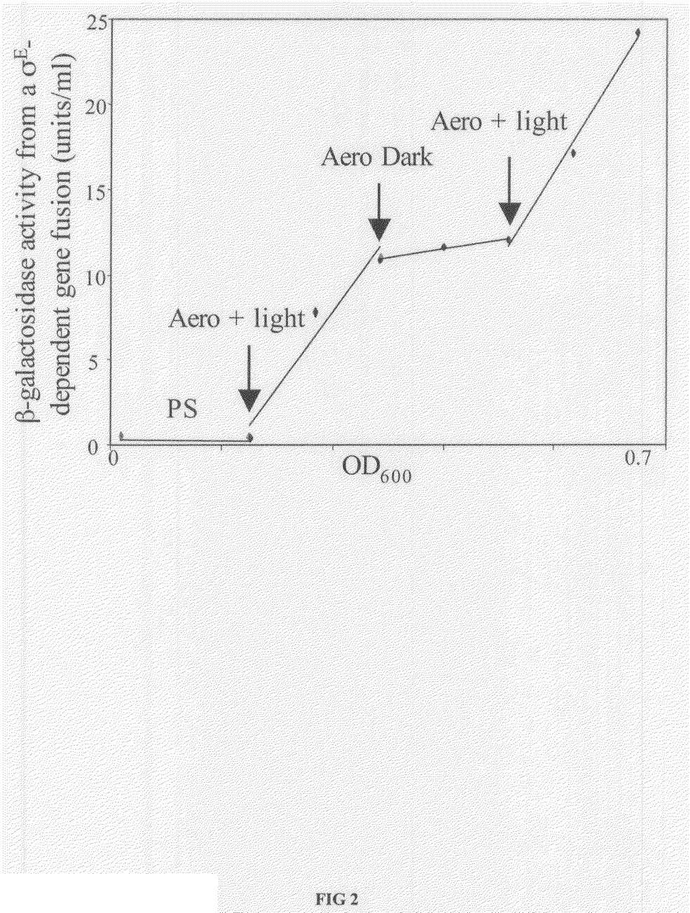

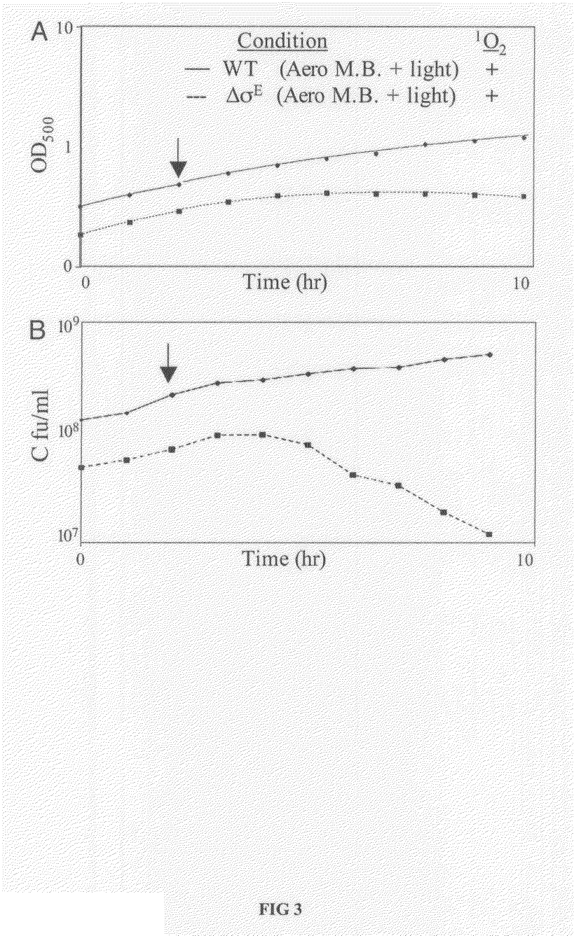

Continued exposure to conditions that generate ¹O₂ in the photosynthetic apparatus are needed to sustain this response: The half-life of ¹O₂ in cells is less than 100 ns and was used to further test if σ^{E} activity was responding to ¹O₂. For example, if increased σ^{E} activity required ¹O₂, then placing photosynthetic cells that had previously been exposed to O₂ in the dark should terminate this transcriptional response. When the cells were shifted to aerobic conditions in the presence of light, we observed an expected increase in the differential rate of β-galactosidase synthesis from the σ^{E}-dependent promoter (˜10-fold, FIG. 2 and Table 3). However, when these cells were placed in the dark (i.e., conditions that allow growth via respiration but prevent ¹O₂ formation), the differential rate of β-galactosidase synthesis decreased ˜9-fold (FIG. 2 and Table 3). Further, an ˜8-fold increase in the differential rate of β-galactosidase synthesis from the σ^{E}-dependent promoter was observed when the same cells were placed back into the light to restore ¹O₂ formation (FIG. 2 and Table 3). This suggests a reversible transcriptional response to ¹O₂ and that increased σ^{E} activity requires continued exposure to ¹O₂.

TABLE 3
Continued exposure to ¹O₂ is required for increased σ^{E} activity.

| Growth | ¹O₂ | Rate |
|---|---|---|
| PS | − | 7 |
| Aero + Light | + | 73 |
| Aero + Dark | − | 8 |
| Aero + Light | + | 63 |

R. sphaeroides σ^{E} activity is increased by formation of O₂ in the absence of the photosynthetic apparatus: If ¹O₂ was responsible for the observed σ^{E} transcriptional response, then other conditions that generate this ROS should also increase σ^{E} activity. To test this hypothesis, one can generate ¹O₂ by illumination of methylene blue in the presence of O₂ to produce a similar response. When aerobically grown WT cells were exposed to 1 μM methylene blue in the presence of light and O₂, cell growth continued and the differential rate of β-galactosidase synthesis from the rpoE P1::lacZ reporter fusion increased ˜20-fold compared to aerobic cells grown in the absence of methylene blue (Table 4). Control experiments indicated there was less than a two-fold increase in the rate of β-galactosidase synthesis when aerobic cultures were exposed to methylene blue in the dark (Table 4). The lack of a comparable increase in σ^{E} activity in aerobic cells exposed to methylene blue in the dark is expected since both light and O₂ are required for this compound to generate ¹O₂.

TABLE 4
Light plus methylene blue increases σ^{E} activity.¹

| Strain | Growth | ¹O₂ | Rate |
|---|---|---|---|
| WT | Aero | − | 5 |
| WT | Aero + light | − | 8 |
| WT | Aero + methylene blue + light | + | 151 |
| WT | Aero + methylene blue (dark) | − | 8 |

¹Differential rates of β-galactosidase synthesis from the σ^{E}-dependent rpoE::lacZ fusion when WT cells are grown aerobically under conditions that either do or do not generate ¹O₂.

For these experiments, cells were grown in the presence of 30% O₂, a condition where pigment-protein complexes of the photosynthetic apparatus are not detectable. Therefore, the transcriptional response to ¹O₂ can occur in cells that either contain or lack the photosynthetic apparatus.

Other ROS do not produce a similar increase in σ^{E} activity: The damaging effects of ¹O₂ on many biomolecules could stimulate the formation of other ROS. To test if other ROS could produce an increase in σ^{E} activity, the differential rate of β-galactosidase synthesis from a rpoE P1::lacZ reporter fusion was monitored in aerobic cells treated with concentrations of hydrogen peroxide (H₂O₂), paraquat (to stimulate superoxide (O₂⁻) formation) or diamide (to alter the oxidation-reduction state of the cytoplasmic thiol pool), previously shown to generate an oxidative stress response in R. sphaeroides. Li K, et al., “Expression of the trxA gene for thioredoxin 1 in Rhodobacter sphaeroides during oxidative stress,” Arch. Microbiol. 180:484-489 (2003). For these experiments, the differential rate of β-galactosidase synthesis was monitored from a control trxA::lacZ reporter fusion, since the trx promoter has previously been shown to respond to oxidative stress in R. sphaeroides.

The addition of paraquat or H₂O₂ to aerobic cells produced increases in the differential rate of β-galactosidase synthesis from the trxA::lacZ reporter gene that are consistent with changes in abundance of trxA transcripts produced by these compounds in previous studies (Table 5). However, the differential rate of β-galactosidase synthesis from the σ^{E}-dependent reporter fusion either decreased (paraquat) or increased no more than 1.2-fold (H₂O₂) when compared to untreated cells (Table 5). Any observed increase in σ^{E} activity in the presence of these ROS was below the 10-fold increase in σ^{E} activity seen when cells are exposed to ¹O₂.

TABLE 5
Other ROS do not increase σ^{E} activity.¹

| | | rpoE P1::lacZ | trxA::lacZ |
|---|---|---|---|
| Addition | ROS | fusion | fusion |
| None | — | 11 | 185 |
| Paraquat | superoxide | 6 | 450 |
| H₂O₂ | peroxide | 13 | 220 |
| Diamide | oxidizes cysteine thiols | 3 | ND |

¹Differential rates of β-galactosidase synthesis from the indicated promoters when WT cells are grown aerobically under conditions that either do or do not generate indicated ROS.
ND—Not Determined.

σ^{E} activity in the presence of diamide was not monitored because previous work has shown that σ^{E} activity does not increase upon exposure to this compound. Based on these results, the transcriptional response observed when ¹O₂ is generated does not occur in the presence of other ROS.

When carotenoids are low, cells require σ^{E} to mount response to O₂: While cells Δσ^{E} cells are unable to mount this transcriptional response to ¹O₂ (FIG. 1 and Table 2), exponential growth of a Aσ^{E} strain continues when a photosynthetic culture is shifted to aerobic conditions in the presence of light (data not shown). This occurs presumably because carotenoids within the photosynthetic apparatus quench ¹O₂. To assess the relative importance of carotenoids and σ^{E} in the presence of ¹O₂, we monitored growth of cells containing low levels of carotenoids in the presence and absence of σ^{E}. For this analysis, cells were grown by aerobic respiration (30% O₂) since they have 20-fold less total carotenoids than photosynthetic cells grown at 10 W/m² (˜10 μg carotenoid/2×10¹⁰ cells compared to ˜200 μg carotenoid/2×10¹⁰ cells, respectively). The use of aerobically grown cells is preferable to studying a carotenoid-minus Δσ^{E} mutant because the lack of carotenoids in such a mutant lowers photosynthetic growth rates.

Exponential growth of aerobically grown WT cells continued after exposure to ¹O₂ (FIG. 3A). In contrast, the number of colony forming units per ml (cfu/ml) of the Δσ^{E} mutant culture decreased ˜10-fold after 8 hours of exposure to ¹O₂ (FIG. 3B). The bacteriocidal effect of ¹O₂ on the Δσ^{E} mutant when carotenoid levels are low shows that both sigma factor activity and carotenoids are critical to viability in the presence of this ¹O₂.

Additional members of the σ^{E} regulon: To identify genes that are part of this transcriptional response to ¹O₂, we compared RNA levels in aerobically grown (30% O₂) WT cells and in a ΔChrR mutant. Because ChrR inhibits σ^{E} activity, one looks for RNA that is more abundant in the ΔChrR mutant. As expected, global gene expression analysis showed an increase (˜12-fold) in rpoE-specific RNA from ΔChrR cells.

RNA from ˜180 genes (˜60 operons) was >3-fold more abundant in cells that contained increased σ^{E} activity (Table 1). In contrast, the ˜35-fold increase in cycA P3 activity that occurs in ΔChrR cells in vivo causes only an ˜1.6-fold increase in total cycA-specific RNA (Table 1). The smaller increase in cycA-specific RNA levels reflects the fact that cycA contains additional strong promoters that are recognized by other sigma factors. This suggests that a global gene expression microarray approach might miss other σ^{E}-dependent genes that also contain multiple promoters.

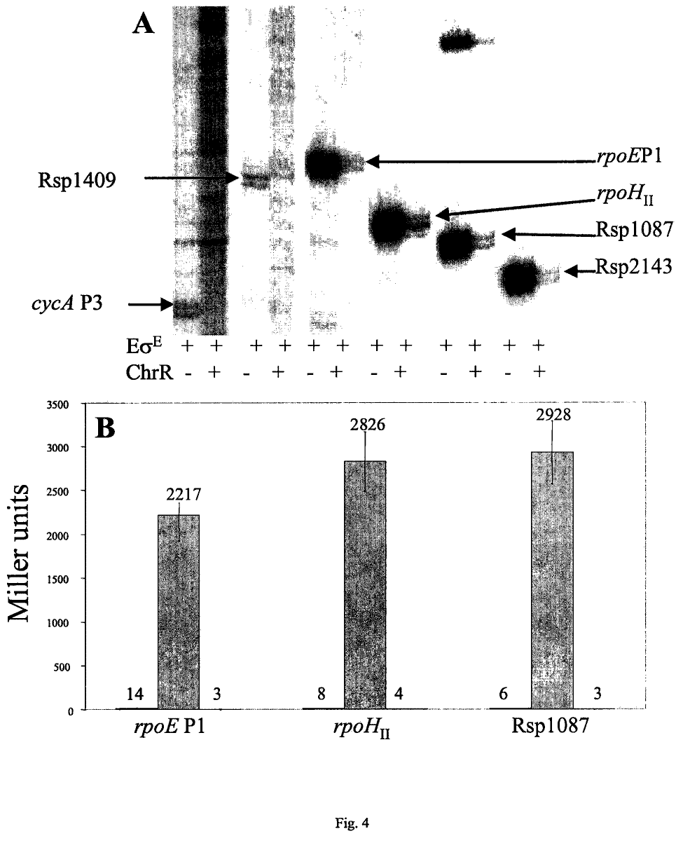

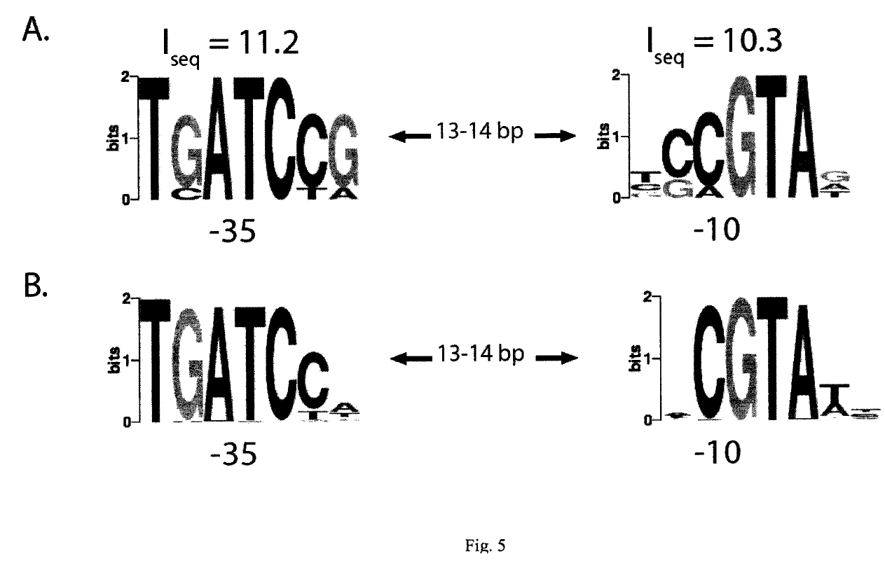

To test if any of these candidate operons contained a σ^{E}-dependent promoter, we tested DNA upstream of the first gene in each of twenty-eight potential operons for transcription by Eσ^{E}. (Table 6) These operons were chosen either because of their increased levels of expression in cells with elevated σ^{E} activity or because of a potential role of their gene products in the photosynthetic apparatus (a source of ¹O₂). It was observed that rpoH_{II}, which encodes one of two R. sphaeroides heat shock sigma factors (Rsp0601), is transcribed by σ^{E}. Production of the rpoH_{II} transcript is inhibited by addition of ChrR, as is the case with other σ^{E}-dependent promoters like rpoE P1 and cycA P3 (FIG. 4A). By these criteria, σ^{E}-dependent promoters are also located upstream of Rsp1087 (which may contain two promoters because different sized σ^{E} transcripts are seen), Rsp 1409, and Rsp2143 (FIG. 4A).

TABLE 6
Operons tested for σ^{E}-dependence.

| | | Fold | Region | σ^{E} | Putative σ^{E}-dependent |
|---|---|---|---|---|---|
| ORF | Description¹ | increase² | tested³ | promoter⁴ | promoter sequence⁴ |
| Rsp0106- | NADH:ubiquinone | 1.7-4.1 | −230 to | − | |
| 0114 | dehydrogenase | | +1 | | |
| Rsp0255- | Bacteriochlorophyll | 1.6-4.1 | −221 to | − | |
| 0261 | synthesis, puf | | +1 | | |
| Rsp0262- | Bacteriochlorophyll | 2-7.8 | −232 to | − | |
| 0263 | synthesis | | +1 | | |
| Rsp0264- | Carotenoid | 1.4 | −240 to | − | |
| 0265 | biosynthesis | | +1 | | |
| Rsp0269- | Carotenoid | 1.4-2 | −217 to | - | |
| 0271 | biosynthesis, tspO | | +1 | | |
| Rsp0284- | Chlorophyll synthesis, | 1.3-4.7 | −201 to | − | |
| 0295 | puhA | | +1 | | |
| Rsp0296 | Cytochrome c₂, cycA | 1.6 | −105 to | + | −88TGATCCN₁₈TAGTGA |
| | | | −42 | | (SEQ ID NO: 4) |
| Rsp0317 | Coproporphyrinogen | 1.5 | −195 to | − | |
| | III oxidase | | +1 | | |
| Rsp0600- | Heat-shock σ factor, | 2.6-38.3 | −209 to | + | −66TGATCCN₁₈TAGTAA |
| 0601 | rpoH₁₁ | | −6 | | (SEQ ID NO: 5) |
| Rsp0896- | Putative glutathione S- | 1-2.9 | −225 to | − | |
| 0898 | transferase | | +1 | | |
| Rsp1025- | DNA polymerase I | 1.2-3.6 | −206 to | − | |
| 1028 | | | −4 | | |
| Rsp1087- | Amine oxidoreductase, | 4.7-180 | −203 to | + | −54TGATCCN₁₈TATCTG |
| 1091 | dehydrogenase | | +1 | | (SEQ ID NO: 6) |
| Rsp1092- | RpoEchrR⁵ | 12.6 | −132 to | + | −130TGATCCN18TAAGAA |
| 1093 | | | −77 | | (SEQ ID NO: 7) |
| Rsp1175 | Methyltransferase | 1.3 | −219 to | − | |
| | | | +1 | | |
| Rsp1277- | CbbXYZ | 1.1-2 | −232 to | − | |
| 1280 | | | +1 | | |
| Rsp1409 | TspO-like regulator | 162 | −223 to | + | −70TCATCCN₁₉TAGCCT |
| | | | +1 | | (SEQ ID NO: 8) |
| Rsp1410- | Putative | 1.6-3.7 | −250 to | − | |
| 1411 | oxidoreductase | | +1 | | |
| Rsp1520 | Histidine sensor | NC | −205 to | − | |
| | kinase, prrB | | −6 | | |
| Rsp1591 | Predicted glutathione | 6.4 | −257 to | − | |
| | S-transferase | | +1 | | |
| Rsp2143- | DNA photoylase, CP- | 2.3-9.9 | −201 to | + | −49TGATCCN₁₈TAAGAG |
| 2146 | FA synthetase | | −2 | | (SEQ ID NO: 9) |
| Rsp2163 | Putative | 1.8 | −406 to | − | |
| | transglycosylase | | −195 | | |
| Rsp2389- | Putative glutathione | 4.5-28.9 | −189 to | − | |
| 2391 | oxidase, histone | | +1 | | |
| | deacytlase | | | | |
| Rsp2683- | Cytochrome | 1.2 | −206 to | − | |
| 2685 | biogenesis, | | +1 | | |
| | endonuclease | | | | |
| Rsp2707- | Pyrophosphate | 1.9 | −206 to | − | |
| 2710 | synthase, Zn- | | +1 | | |
| | dependent protease | | | | |
| Rsp3075- | Uncharacterized | 11-16.6 | −185 to | − | |
| 3076 | conserved proteins | | +1 | | |
| Rsp3117 | Hypothetical protein | NC | −189 to | − | |
| | | | +1 | | |
| Rsp3162- | Probable | 3.7-20.3 | −237 to | − | |
| 3164 | oxidoreductase | | +1 | | |
| Rsp3210, | Quinol oxidase, | 6-7.6 | −195 to | − | |
| 3212 | qxtAB | | +1 | | |
| Rsp3272- | ATP transporter, | 3.1-12.2 | −212 to | − | |
| 3274 | glutathione | | +1 | | |
| | degradation | | | | |
| Rsp3310 | Short-chain | 9.1 | −199 to | − | |
| | dehydrogenase | | +1 | | |

¹Function known or predicted by genome annotation. Genes were chosen based on increased RNA abundance in cells that have elevated σ^{E} activity or for their known role in photosynthetic growth.
²Increase in RNA abundance from comparing transcript levels in WT and ΔChR cells. Data has been deposited at GEO under accession number GSE2219. NC- no change.
³Coordinates are numbered relative to the start site of translation.
⁴Based on the ability to detect a σ^{E}-dependent transcript in vitro (see FIG. 4A).

Each gene is predicted to be part of a polycistronic operon that encodes uncharacterized proteins. The level of transcripts produced from the rpoH_{II}, Rsp1087 and Rsp2143 promoters are comparable to that of rpoE P1 (within 1.1-fold), suggesting that these 4 promoters are of similar strength. In contrast, the abundance of the σ^{E}-dependent transcript produced by Rsp1409 in vitro is comparable to the σ^{E}-dependent promoter, cycA P3, which has ˜80-fold less activity than rpoE P1.

The same putative rpoH_{II} and Rsp1087 promoter regions were fused to lacZ to test for σ^{E}-dependent activity in vivo. Expression was not detectable from these reporter fusions in WT R. sphaeroides cells, but it was comparable to that of rpoE P1 in ΔChrR cells (FIG. 4B). In addition, activity from the rpoH_{II} and Rsp1087 promoters was not detectable in Δσ^{E} cells (FIG. 4B). This suggests that transcription from this promoter region is dependent solely on σ^{E}, as is the case for rpoE P1.

### Example 2

### Inhibiting a Microbial ¹O₂ Response

Generation of ChrR mutants to irreversibly bind to σ^{E}. The N-terminal anti-sigma domain of ChrR (ChrR-ASD) appears important in binding between ChrR and σ^{E} (data not shown). The skilled artisan is familiar with methods for delivering genetically engineered antimicrobial agents to microbes by phage therapy. Westwater C, et al., “Use of genetically engineered phage to deliver antimicrobial agents to bacteria: an alternative therapy for treatment of bacterial infections,” Antimicrob. Agents Chemother. 47:1301-1307 (2003), incorporated herein as if set forth in its entirety. Phage delivery systems are advantageous because they allow for targeting specific bacterial cells at a high frequency. Accordingly, a phage DNA is modified to contain a coding sequence that codes for at least amino acids 1-85 from GenBank Accession No. AAB17905 (SEQ ID NO:1), which discloses the R. sphaeroides full-length ChrR sequence. The N-terminal portion of ChrR encoded by this construct is sufficient to irreversibly bind zinc and σ^{E}. However, cells containing this or similar N-terminal ChrR variants are not able to mount a response to ¹O₂, resulting in a condition where cells have increased sensitivity to this reactive oxygen species.

Bacterial cells are grown under standard culture conditions. Once an adequate concentration of bacterial cells is present, they are infected with a phage modified to express at least amino acids 1-85 of SEQ ID NO:1. Following exposure to the phage, oxidative stress ensues, but the cells do not express genes regulated by σ^{E}. Consequently, the concentration of bacterial cells decreases.

Alternatively, bacterial cells are infected with a phage modified to express at least amino acids 1-85 of SEQ ID NO:1. They are then grown under standard culture conditions; however, the concentration of bacterial cells does not increase upon oxidative stress because the cells do not express genes regulated by σ^{E}.

Other methods for reducing availability of σ^{E} can include using RNAi directed against σ^{E}, mutating the promoter that directs transcription of σ^{E} (see Newman et al. (1999), supra), and engineering the cells to put σ^{E} under control of a regulatable promoter or repressor.

### Example 3

### Generating Commodity Chemicals in Phototrophs in the Presence of ¹O₂

Bacterial cells with a modified ChrR that cannot bind σ^{E} are grown under standard culture conditions. However, growth and, consequently, production of a commodity chemical are increased because the cells are protected against the deleterious effects of ¹O₂.

Likewise, bacterial cells with a modified σ^{E} that cannot be bound by ChrR are grown under standard culture conditions. However, growth and consequently production of a commodity chemical are increased because the cells are protected against the deleterious effects of ¹O₂.

### Example 4

### Modifying Plants Lipid Bilayers for Protection During ¹O₂ Challenge

Methods of manipulating plant genes are known to the skilled artisan. For example, Constabel C, et al., “Transgenic potato plants overexpressing the pathogenesis-related STH-2 gene show unaltered susceptibility to Phytophthora infestans and potato virus X,” Plant Mol. Biol. 22:775-782 (1993), incorporated herein by reference as if set forth in its entirety. Accordingly, a plant is modified such that the plant exhibits a high level of cyclopropane-fatty-acyl-phospholipid synthase (CfaS) relative to an unmodified plant. The plant is grown under standard conditions; however, growth is increased because the plant is protected against the deleterious effects of ¹O₂.

### Example 5

### Producing Peptides in Phototrophs During ¹O₂ Challenge

Methods of inserting a gene of interest into a plasmid are known to the skilled artisan. Schilke & Donohue, supra. A gene encoding a product of interest is inserted into a plasmid under regulation of a σ^{E}-dependent promoter selected from the consensus sequence (SEQ ID NO:3) in FIG. 5. Bacterial cells containing a plasmid having a gene regulated by a σ^{E}-dependent promoter selected from the consensus sequence (SEQ ID NO:3) in FIG. 5 are grown under standard culture conditions. However, cell growth and production of the gene product are increased because ¹O₂ increases transcription from the promoter.

### Example 6

### Identifying σ^{E}-Dependent Genes in R. Sphaeroides

Using a combination of clustering analysis, promoter motif predictions, and functional assays, the inventors revealed that σE directly transcribed 9 operons with a total of 15 genes and that the promoter motif of these operons has a high information content (as defined in paragraph [00099]). The R. sphaeroides rpoEchrR operon, which encodes σ^{E} and ChrR, contains a σ^{E}-dependent promoter. Expression from this promoter increases in cells with a mutated ChrR (ΔChrR) due to the high σ^{E} activity in those cells. To identify potential members of the σ^{E}-ChrR regulon, the inventors clustered transcript levels from 67 R. sphaeroides global gene expression datasets collected from the Gene Expression Omnibus database, available at the NCBI GEO website, as of May 2005. The datasets contain RNA abundance measurements from 67 GeneChip Custom Express microarrays (Affymetrix, Santa Clara, Calif.) obtained from wild-type or mutant strains grown in a succinate-based minimal medium under various conditions (GSM1670, GSM1671, GSM1672, GSM1673, GSM2416, GSM2417, GSM2418, GSM2419, GSM2420, GSM2421, GSM2422, GSM2423, GSM2424, GSM2425, GSM2426, GSM2427, GSM2429, GSM2430, GSM2450, GSM25295, GSM25296, GSM25297, GSM25298, GSM25299, GSM25300, GSM25301, GSM25302, GSM25303, GSM26242, GSM26243, GSM26244, GSM26245, GSM26260, GSM26262, GSM26263, GSM26265, GSM26266, GSM27348, GSM27349, GSM27350, GSM27351, GSM27352, GSM27353, GSM3030, GSM3031, GSM3032, GSM3258, GSM3260, GSM3262, GSM3272, GSM3273, GSM3274, GSM38774, GSM38775, GSM38776, GSM38777, GSM38778, GSM38779, GSM38780, GSM38781, GSM38782, GSM38783, GSM38810, GSM40560, GSM8107, GSM8108, and GSM8109). RNA levels in all datasets were normalized using the Robust Multichip Average method to log₂ scale. Irizarry et al., Biostatistics 4:249-264 (2003). To limit spurious correlation in the clustering steps, the inventors selected only those RNA species that were twofold or more abundant (FDR=0.1) in ΔchrR cells compared to wild type cells as determined by EBarrays software (Kendziorski et al., Stat Med. 22:3899-3914 (2003)). Approximately 100 loci were selected based on these criteria. Hierarchical clusters were constructed with the R statistical software environment, available from the Department of Statistics and Mathematics of the WU Wien, using (1-Pearson's correlation coefficient)/2 as the distance between expression patterns and the “complete” method for cluster linkage. The Pearson's correlation coefficient indicates the degree of association between two variables. A positive correlation value indicates positive association and a negative correlation value indicates negative or inverse association.

The Pearson's correlation coefficient between the abundance of rpoE transcripts and that of chrR transcripts was high (0.95) because these two genes are cotranscribed. In addition, the correlation coefficient between transcript levels for rpoE and another known σ^{E} target gene (rpoH_{II}) was 0.88, suggesting that clustering genes with expression patterns that correlated with that of rpoE could predict other potential members of the σ^{E}-ChrR regulon. RNA abundance levels from the resulting 110 genes were clustered in a hierarchical tree based on the Pearson's correlation coefficient of their respective transcript patterns. In this tree, a cluster of transcript patterns containing rpoE and some other known σE target genes was identified, indicating that this method successfully predicts other members of the σE-ChrR regulon. Likewise, genes that are likely to belong to the same transcription unit, based on computational predictions of R. sphaeroides operons, were present in the rpoE-containing transcript cluster, providing further confidence in the predictive nature of the output. This rpoE-containing cluster contains one gene that had not previously been identified as a potential σE target (RSP1852), suggesting the existence of additional yet-to-be-identified members of this regulon.

The inventors hypothesized that operons directly transcribed by σE should contain a conserved promoter motif. To test this hypothesis, DNA sequences upstream of the potentially clustered operons were queried for a common sequence element. The BioProspector algorithm was used to search for a conserved bipartite motif within 300 by upstream of the predicted translation starting site in sets of candidate promoter sequences. The parameters were set to search for two blocks of 6 by and for a gap between 13 by and 16 bp. Sequence logos were generated using WebLogo, available at the Berkeley University, California website. Sequence logos are generated by aligning sequences and graphically representing the respective frequency of a nucleotide at a given position. Crooks et al., Genome Research, 14:1188-1190 (2004); Schneider and Stephens, Nucleic Acids Res. 18:6097-6100 (1990), each of which is incorporated herein by reference as if set forth in its entirety. The taller a nucleotide symbol, the greater its frequency at a particular position. Also, the taller the overall stack of nucleotide symbols, the greater the conservation at a particular position of the aligned sequences.

A conserved motif was found upstream of all the potential operons contained within the rpoE-containing cluster (FIG. 5A). This motif contains two high-information-content regions (as defined in paragraph 00099) separated by a variable spacer sequence, which is typical of the −35 and −10 regions recognized by group IV bacterial σ factors. All but one of the known or predicted σE promoters have 13 by between their putative −10 and −35 elements; the exception (RSP1409) has a 14-bp spacer in its putative promoter.

To search for additional candidate members of the σE-ChrR regulon that were not detected by analyzing the global gene expression patterns, a library of putative R. sphaeroides promoters was queried for sequences related to the conserved sequence motif. The conserved sequence motif was converted into a position-specific weighted matrix (PSWM), which represents motifs in biological sequences, that was used to score the promoter library based on the content information of the best match in each sequence. To relax the stringency of the query, spacer lengths of 13 by or 14 by between the −35 region and the −10 region were allowed without penalty. Six of the best fifteen matches (Table 7) were known σE target operons and one was a candidate σE promoter upstream of a gene within the rpoE-containing cluster (RSP1852). None of the promoter regions for any of the other 110 differentially expressed genes analyzed above contained a match to the motif with a score ≧75% of the maximum. Thus, it appears that the other differentially expressed operons are not likely to contain σE promoters.

TABLE 7
Candidate and confirmed σ^{E} target operons and their putative promoters.

| | | | Normal- | | | | Distance | Corre- |
|---|---|---|---|---|---|---|---|---|
| | | | ized | | | | to start | lation |
| Gene ID | Name | Score | score | −35 | Spacer | −10 | codon | coefficient |

A. Putative promoters identified from the sequence analysis of a
library of promoter regions

| | | | | | | | | | |
|---|---|---|---|---|---|---|---|---|---|
| | RSP1087- | | 20.14 | 1.00 | TGATCCG | ccttgggcgacag | TCCGTAT | −54 | 0.91 |
| | 1091 | | | | | | | | |
| | RSP0601 | rpoH11 | 19.50 | 0.97 | TGATCCG | gacatgtgttttt | TCCGTAG | −66 | 0.88 |
| | RSP2143- | phrBcfaS | 18.95 | 0.91 | TGATCCG | ggaagcgggcccg | CGCGTAA | −49 | 0.85 |
| | 2144 | | | | | | | | |
| | RSP1092- | rpoEchrR | 18.32 | 0.91 | TGATCCA | gactggcccggcc | GCCGTAA | −130 | 1 |
| | 1093 | | | | | | | | |
| | RSP1409 | | 18.14 | 0.90 | TCATCCG | ccggagccgccttc | TGCGTAG | −71 | 0.84 |
| ★ | RSP1852 | | 18.06 | 0.90 | TGATCTG | aaccgtcgcttaa | CCCGTAT | −103 | 0.93 |
| | RSP6076 | | 17.89 | 0.89 | TGATCTT | caagtgagacccga | TCCGTAA | −43 | NA |
| | RSP3284 | | 17.51 | 0.87 | TGATCCG | gaggtcgggcctc | TCCGAAG | −103 | −0.30 |
| | RSP0296 | cycA | 16.83 | 0.84 | TGATCCG | gaacgcgcggccc | GCAGTAG | −88 | −0.12 |
| | RSP4129 | | 16.37 | 0.82 | TGATCTG | aaactaaagcttt | GCCATAT | −180 | −0.22 |
| ★ | RSP6222 | | 16.26 | 0.80 | TGATCTT | catggggatatct | CCCGTAG | −72 | NA |
| | RSP1207 | hslO | 16.15 | 0.80 | CGATCCG | cccgcacggggtc | GCCGTAT | −110 | 0.58 |
| ★ | RSP3336 | | 15.66 | 0.78 | AGATCTG | acgtgaacaagat | ACCGTAA | −172 | −0.25 |
| | RSP1521 | | 15.72 | 0.78 | TGATCCA | gacctgatccggc | GCCGGAT | −140 | −0.20 |
| | RSP0357 | | 15.72 | 0.78 | TGATCCA | gctcgccgccatc | GCCGTGA | −183 | 0.63 |

B. Putative additional promoters identified within the regions significantly
enriched in the ChIP-chip experiment using anti-σ^{E} antibodies

| | | | | | | | | |
|---|---|---|---|---|---|---|---|---|
| RSP2324 | | 16.80 | 0.85 | TGATCCG | gcgccgattgca | ATCGTAG | −434 | 0.03 |
| RSP2978 | mrcA | 16.54 | 0.84 | AGATCCG | gctgatcgtcggc | GGCGTAT | NA | NA |
| RSP1955 | | 16.44 | 0.83 | TGGTCCG | gagcggtctcgcg | TGCGTAG | NA | NA |
| RSP1222 | ham1 | 15.99 | 0.81 | AGATCCA | gcaccggctggcc | CGCGTAG | NA | NA |
| RSP2047 | | 15.87 | 0.80 | GCATCCG | gttacctccttgc | TGCGTAT | +39 | −0.05 |
| RSP3101 | | 15.47 | 0.78 | CGATCCA | ccttccatcatct | TTCGTAT | −68 | −0.24 |
| RSF1612 | | 14.79 | 0.74 | CGATCAG | ctggcccgcag | CCTGTAG | NA | NA |
| RSP4003 | dhaL | 14.58 | 0.74 | CGATCCA | gatggtcttcagc | TGGGTAT | NA | NA |
| RSP2401 | | 13.67 | 0.69 | TGGACCG | gatgcgactctcc | ACCGTAG | −56 | 0.27 |
| RSP2793 | | 13.66 | 0.69 | GGATTTG | ccatggaaaacgag | CGCGTAA | NA | NA |
| RSP2940 | | 12.38 | 0.63 | CCATCAG | ccgggcggcggcat | CCCGCAT | NA | NA |
| RSP3007 | | 11.05 | 0.56 | CAATCTC | gaaggaatgttca | GGCGTAT | NA | NA |

Previously identified σ^{E} target operons/genes 6·9 are shown in bold; starred genes were shown to be members of the σ^{E}-ChrR regulon in this study. Promoters upstream of the remaining candidate genes/operons promoters failed to produce detectable levels of transcripts with reconstituted Eσ^{E} in vitro.
The normalized score is relative to the maximal score that can be obtained from the constructed PSWM for the σ^{E} promoter motif. The correlation coefficient represents the Pearson's correlation coefficient of each transcript level pattern relative to the rpoE transcript level pattern from the expression microarray dataset used in this study.

To determine if the candidate sequences (Table 7) contain functional σE promoters, the regions spanning from −200 by to +80 by relative to the predicted transcription initiation site were analyzed for function in vitro and in vivo, as described in Example 1. The inventors determined the relative strength of these candidate promoters by comparing their activity to known GE target genes and analyzed the ability of ChrR to inhibit the candidate promoter's function.

In vitro transcription using R. sphaeroides core RNA polymerase reconstituted with recombinant σE produced a product of the predicted size from the candidate RSP1852 promoter at a level similar to that obtained from the strong σE-dependent promoter (RSP1092) upstream of rpoE. Addition of the anti-σ factor ChrR prevented the accumulation of the σE transcript from the known σE SP1092 promoter and the candidate RSP1852 promoter. In contrast, the levels of transcript obtained from the candidate RSP3336 and RSP6222 promoters were lower, and detection of these products required a σE concentration that was 5- to 10-fold (250-500 nM recombinant protein) in excess over R. sphaeroides core RNA polymerase (50 nM). Similar increases in the concentration of σ^{E} do not generate a higher amount of product from strong promoters such as RSP 1092 in multiple-round transcription assays under these conditions. Thus, DNA binding by Eσ^{E} holoenzyme may be a limiting kinetic step for transcription at the RSP3336 and RSP6222 promoters. The inventors could not assess whether ChrR inhibits transcription from the RSP3336 and RSP6222 candidate promoters because the anti-σ factor could not be obtained at concentrations sufficiently high to inhibit all the GE in the assay. All other candidate promoters shown in Table 7 failed to produce detectable in vitro transcripts, regardless of the levels of σ^{E} added to core RNA polymerase (data not shown).

Specific bases at these positions of the −10 and −35 elements are associated with σ^{E}-dependent transcription. The tested promoters active with σ^{E} in vitro contain a GTA motif in their −10 region, whereas those that were inactive in this assay have one or more substitutions to any other nucleotide in this motif. The RSP1207 promoter, also not transcribed by σ^{E} in vitro, contains a GTA motif in its putative −10 region but contains a substitution in place of the first T of the −35 region that is conserved in all active promoters.

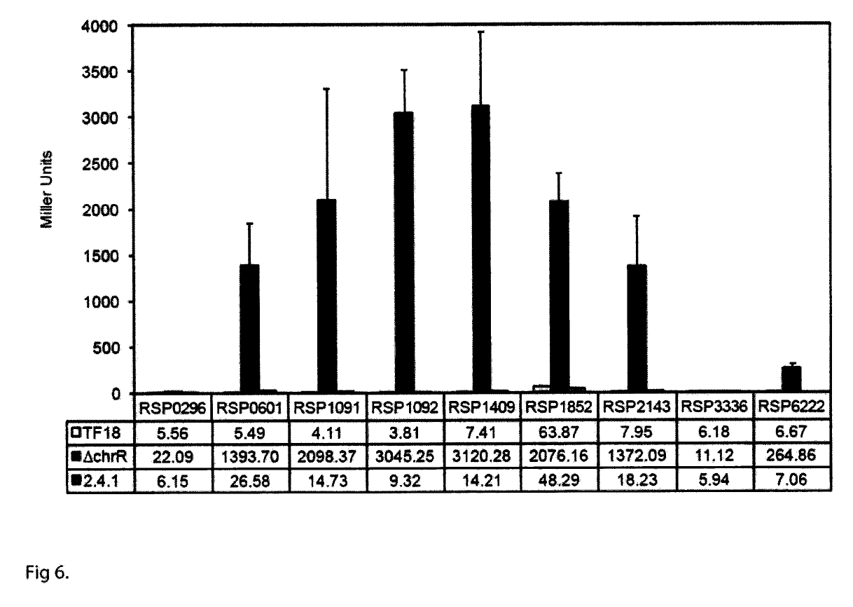

To test for promoter activity in vivo, these and other candidate promoter sequences were fused to a promoterless β-galactosidase gene on a low-copy plasmid. The activity of these reporter genes was tested in wild-type R. sphaeroides cells (low σ^{E} activity), ΔChrR cells (high σ^{E} activity), and cells lacking the rpoEchrR operon (no σ^{E} activity). Consistent with the results of in vitro transcription assays, the inventors observed high reporter gene activity in ΔChrR cells from known σ^{E} target genes (RSP601, RSP1091, RSP1092, RSP1409, and RSP2143) and RSP1852, while both RSP6222 and RSP3336 showed lower activity (FIG. 6). None of these promoters was active in AGE cells, suggesting that function under these conditions required a functional rpoE gene. RSP6076, RSP4129, RSP3284, RSP1207, RSP1521, and RSP0357 (table 7) did not show σE-dependent promoter activity in either assay (data not shown).

To identify possible additional members of the σE-ChrR regulon, the inventors assessed genome-wide interactions of GE or the β′ subunit of RNA polymerase with DNA using chromatin immunoprecipitation and microarray (ChIP-chip) analysis. For immunoprecipitation, specific polyclonal antibodies were used to enrich DNA bound by σE or the (3′ subunit in ΔChrR cells (high σE activity). ChIP-chip hybridization was performed essentially as described in Lee et al., Nat. Protoc. 1:729-748 (2006), incorporated herein by reference as if set forth in its entirety. R. sphaeroides ΔchrR cells (increased σ^{E} activity) were grown by bubbling 500 ml of cell culture with 1% CO₂, 30% O₂, and 69% N₂. At midexponential phase (˜2×10⁸ colony-forming units/ml), formaldehyde and sodium phosphate were added to a final concentration of 1% and 10 mM, respectively. This mixture was incubated at 30° C. for 4 minutes before glycine was added to 100 mM, and the solution was placed on ice for 30 minutes with gentle agitation to quench excess formaldehyde. Cells were centrifuged at 3000 g, washed twice with chilled phosphate-buffered saline, centrifuged, and flash-frozen at −80° C. About 2×10¹⁰ cells were suspended in 0.5 ml of 100 mM Tris (pH 8.0), 300 mM NaCl, 2% Triton X-100, and 1 mM phenylmethylsulfonyl fluoride, and sonicated eight times for 20 seconds with a Branson Sonifier (Branson Ultrasonics Corp., Danbury, Conn.) set to level 6 and 50% output using a 3-mm microtip. A mixture of micrococcal nuclease (50 U) and RNase A (0.5 μg) in 200 μM CaCl₂, 1.2 mM KCl, 6 mM sucrose, and 10 μM DTT was added. The mixture was incubated for 1 hour at 4° C.; and then nuclease activity was inhibited by adding 10 mM ethylenediaminetetraacetic acid (EDTA). Cell debris was removed by centrifuging for 10 minutes at 12,000 g, and an aliquot was removed to analyze DNA fragmentation by agarose gel eletrophoresis (desired size of ˜200-1000 by with enrichment for ˜500-bp molecules). The supernatant was incubated with gentle mixing with 20 μl of Staphylococcus aureus protein A Sepharose beads (Sigma-Aldrich, St. Louis, Mo.) for 3 hours at 4° C. as pretreatment to remove potential nonspecific binding to the beads. After the beads had been removed by centrifugation (5 minutes at 3000 g), one-tenth of the sample was removed and used as non-antibody-treated control. Two microliters of the anti-R. sphaeroides σ^{E} rabbit polyclonal antibody serum was added, and the mixture was incubated overnight at 4° C. with gentle mixing before being incubated with protein A Sepharose beads (30 μl) for 2 hours at 4° C. The beads were recovered by centrifugation and then washed once at 4° C. with 250 mM LiCl, 100 mM Tris (pH 8.0), and 2% Triton X-100, followed by two washes in 600 mM NaCl, 100 mM Tris (pH 8.0), and 2% Triton X-100; two washes in 300 mM NaCl, 100 mM Tris (pH 8.0), and 2% Triton X-100; and two washes in TE buffer (10 mM Tris pH 8.0 and 1 mM EDTA). Protein-DNA complexes were eluted from the beads by incubation at 65° C. for 30 minutes in 50 mM Tris (pH 8.0), 10 nM EDTA, and 1% sodium dodecyl sulfate. The beads were removed by centrifugation, and protein-DNA cross-linking was reversed by incubating the samples for 12 hours at 65° C.

DNA was purified using the QIAquick PCR Purification Kit (QIAGEN, Inc., Valencia, Calif.) and amplified via ligation-mediated PCR. DNA fragments were treated with T4 DNA polymerase, purified by phenol extraction and ethanol precipitation (adding 20 μg of glycogen), and ligated with T4 DNA ligase to 4 μM annealed oligonucleotide linkers (oJW102: GCGGTGACCCGGGAGATCTGAATTC; oJW103: GAATTCAGATC) at 16° C. overnight. DNA was ethanol-precipitated before amplification (22 cycles; annealing temperature of 60° C.) with Taq DNA polymerase (New England Biolabs) and 1 μM oJW102 linker (50-μl reaction). The amplification reaction was repeated using 15 μl of the first amplification reaction product as template. After the products had been purified using a QIAquick Kit (QIAGEN), triplicates of control and sample DNA (concentrated to ˜250 ng/μl) were pooled to obtain ˜4-μg quantities and hybridized to a custom-made NimbleGen microarray (NimbleGen Systems, Madison, Wis.). For each antibody, triplicate microarrays were analyzed.

The custom-made NimbleGen microarray used for ChIP-chip analysis was designed to tile the R. sphaeroides 2.4.1 genome (two chromosomes and five plasmids) with overlapping isothermal probes that ranged from 35 to 65 bases, with an average spacing of 12 bp. The probes on the array alternate between coding and noncoding DNA sequences.

For analysis of each ChIP-chip microarray, quantile normalization was used to obtain the same empirical distribution across the Cy3 and Cy5 channels and across arrays to correct for dye intensity bias and to minimize microarray-to-microarray absolute intensity variations. Bolstad et al., Bioinformatics 19:185-193 (2003). The log₂ of the ratio of experimental signals (Cy3) to control signals (Cy5) was calculated. The data from the biological replicates were averaged for visualizations in SignalMap 1.9 software (NimbleGen Systems). Regions of the genome enriched for occupancy by σ^{E} were identified using TAMALPAIS, available from the UC Davis Genome Center, at p≦0.01 for a threshold set at the 98th percentile of the log₂ ratio for each chip. Only enriched regions that achieved the specified statistical significance in all three replicates were considered. The peaks were ranked by order of intensities calculated from the average of the 10 highest consecutive probe signals for each peak.

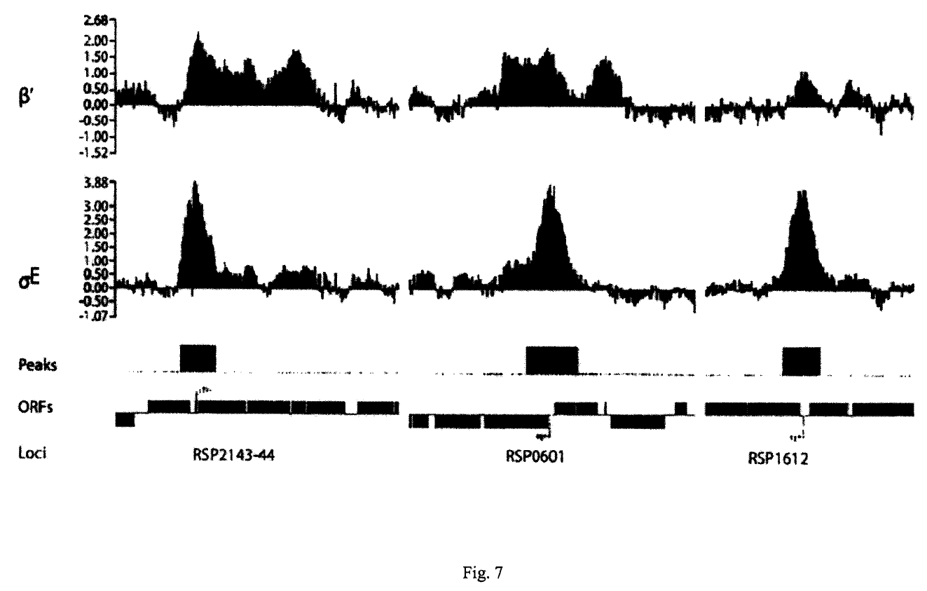

Regions containing strong σ^{E}-dependent promoters (RSP2143-2144, RSP0601, RSP1091-1087, RSP1092-1093, RSP1852, and RSP1409) were significantly enriched by immunoprecipitation of (Table 8). In addition, each of these operons showed enrichment for the β′ subunit of RNA polymerase (FIG. 7), indicating that they were actively transcribed in ΔChrR cells (high σ^{E} activity). Furthermore, maximal occupancy by σ^{E} coincided with the position of the conserved motif (FIG. 5A). Because no evidence for σ^{E} or RNA polymerase interaction was detected at promoters that had very low activity in vitro or in vivo (RSP0296, RSP3336, and RSP6222; FIG. 6), other weak σ^{E}-dependent promoters not revealed by this assay may exist.

Most of the σE occupancy sites detected by this assay that were not also detected by the clustering or motif-scanning analysis, were within protein coding sequences or large intergenic regions (Table 8). Further, there is no evidence that these other σ^{E} occupancy sites are functional σ^{E}-dependent promoters in vivo because most of the conserved motifs were oriented in the opposite direction of the nearby open reading, RNA polymerase occupancy could not be found beyond the putative σ^{E} binding sites (with the exception of RSP1612 (FIG. 7), RSP3832, and RSP2401), and none of the open reading frames neighboring the 13 other regions of σ^{E} occupancy showed increased expression levels in the global gene expression dataset when σ^{E} activity was high. However, additional σ^{E}-dependent promoters directing the transcription of small nonannotated open reading frames or small RNA may exist. Alternatively, regions that appear untranscribed but are enriched for σ^{E} may represent RNA polymerase complexes poised in promoters that require different environmental conditions or additional proteins to be actively transcribed.

### Example 7

### Phylogentically Analyzing σ^{E}-ChrR Across the Bacterial Kingdom

The inventors identified a set of genes that potentially constitute the core of σ^{E}-dependent biological response to ¹O₂ and that is potentially conserved across distantly related bacterial species. To determine if the response to singlet oxygen is regulated in other bacteria in a manner similar to the R. sphaeroides σE-ChrR regulon system, the inventors tested for the presence of this response across the bacterial kingdom. The inventors analyzed the genomes of 84 bacteria that contain a gene encoding a group IV alternative a factor adjacent to one coding for a ChrR homolog. The analysis was limited to 73 bacteria with almost complete genome sequences to probe for both σE-ChrR as well as members of the σE-ChrR regulon. All genomic DNA sequences were obtained from the Integrated Microbial Genomes system on Jan. 10, 2007, available at the Department of Energy Joint Genome Institute website. Groups of orthologous genes were determined using the OrthoMCL software set to the default parameters. The protein sequences of orthologous gene products were aligned with CLUSTAL W, a multiple sequence alignment computer program, using the default parameters. Phylogenetic trees were constructed from the protein sequence alignments with the MrBayes 3.1.2 software over 4×10⁶ generations (first 25% as burn-in) using the General Time-Reversible model including an estimated proportion of invariable sites and a γ-shaped distribution of rate variation across sites. Ronquist, F. & Huelsenbeck, J. P. 2003 Bioinformatics 19, 1572-1574.

TABLE 8
Regions occupied by Eσ^{E} in vivo as determined by ChIP.

| | Genomic | | | | |
|---|---|---|---|---|---|
| | coordinates | | σ^{E} | | |
| Scaffold | Start | End | enrichment | β′enrichment | Associated locus |
| Chromosome 1 | 744077 | 745110 | 35.09 | 19.02 | RSP2143, upstream |
| Chromosome 1 | 2342719 | 2344030 | 34.25 | 14.78 | RSP0601, upstream |
| Chromosome 1 | 210584 | 211536 | 33.71 | 7.80 | RSP1612-1613, intergenic antisense |
| Chromosome 2 | 28320 | 29366 | 31.08 | 8.00 | RSP3832-3833, intergenic antisense |
| Chromosome 1 | 2848771 | 2853927 | 30.86 | 13.37 | RSP1091-1092, upstream |
| Chromosome 1 | 445724 | 446747 | 26.36 | 10.93 | RSP1852, upstream |
| Plasmid P002 | 44685 | 45230 | 25.65 | 2.05 | RSP4003, within coding sequence |
| | | | | | antisense |
| Chromosome 2 | 145759 | 146248 | 20.57 | 6.21 | RSP3101, upstream |
| Chromosome 1 | 642229 | 642880 | 19.11 | 5.34 | RSP2047, upstream |
| Chromosome 1 | 3186065 | 3186861 | 18.73 | 1.05 | RSP1409, upstream |
| Chromosome 1 | 1030027 | 1030565 | 17.55 | 17.09 | RSP2401, upstream |
| Chromosome 1 | 1668034 | 1668547 | 16.92 | 4.58 | RSP2978, within coding sequence |
| | | | | | antisense |
| Chromosome 1 | 2994575 | 2994971 | 14.90 | 0.66 | RSP1221, within coding sequence |
| | | | | | antisense |
| Chromosome 1 | 946459 | 946982 | 14.55 | 1.07 | RSP2324, upstream |
| Chromosome 1 | 1528441 | 1528994 | 14.36 | 0.44 | RSP2793, within coding sequence |
| | | | | | antisense |
| Chromosome 1 | 1619624 | 1620253 | 13.97 | −1.69 | RSP2940, within coding sequence |
| | | | | | antisense |
| Chromosome 1 | 551623 | 552034 | 11.94 | 3.52 | RSP1955-1956, intergenic antisense |
| Chromosome 2 | 42996 | 43226 | 11.53 | 0.47 | RSP3007-3008, intergenic antisense |

σ^{E} enrichment represents the average of the log₂ ratio of the 10 best consecutive probes in each selected region.
The β′ signal represents the average of the log₂ ratio of the same probes from the β′ immunoprecipitation experiment (see Materials and Methods).
Previously validated σ^{E}-dependent promoters are indicated in bold.

A test promoter library for each bacterium was constructed by extracting 300 by upstream of each protein coding sequence. Each test promoter sequence was scanned for its best match to the defined PSWM according to its information content (I_{seq}):

$$\underline{{I}_{seq} = {\sum }_{i} {\sum }_{b} {f}_{b , i} {log}_{2} \frac{{f}_{b , i}}{{p}_{b}}}$$

where i is the position in the matrix, b is one of the four bases, f_{b,i} is the observed frequency of each base at that position, p_{b} is the background frequency of each base in the target genome (p_{A}+p_{T}=0.31 and p_{G}+p_{C}=0.69 for R. sphaeroides). Higher information content is designated by values closer to 1, lower information content is designated by lower values. The skilled person will appreciate that this is an empirical determination made on a case-by-case basis, such that no bright line distinctions between the two are appropriate, although the skilled artisan can evaluate the values as needed. The motif was defined by two matrices (6 by for the −35 region and 4 by for the −10 region). Alternative spacer lengths of 15-17 by were considered without penalties. Promoter sequences containing a match of at least 0.75 times the maximal information content that could be obtained from the PSWM were considered positive and assigned a value of 1.0. Genes directly downstream and transcribed in the same direction of genes with a positive promoter were assigned a score of 0.8. The next gene downstream was assigned a score of 0.6 and so on, until either the end of a potential operon was reached (five genes) or another gene transcribed in the opposite was found. The score for the each group of orthologous gene products was calculated by summing individual gene scores.

Bacteria having σE-ChrR homologs were distributed almost evenly between α-proteobacteria and γ-proteobacteria, with only one encoded by a β-proteobacterium (Acidovorax anenae subsp. citrulli) or a 8-proteobacterium (Myxococcus xanthus).

To test for lateral transfer of the σE-ChrR structural genes, the inventors constructed a species tree based on the amino acid sequence of RuvB, RpoD, and GyrB. These proteins were selected to construct the species tree because they are encoded by single copy genes that are only rarely subject to lateral gene transfer in the selected bacteria. The phylogenetic trees constructed with each protein supported the notion that no lateral gene transfer occurred. The inventors concatenated the RuvB, RpoD, and GyrB protein sequences to build a species phylogenetic tree. Individual phylogenetic trees constructed for σE and ChrR also suggested their coinheritance, so their amino acid sequences were concatenated to build a σE-ChrR phylogenetic tree.

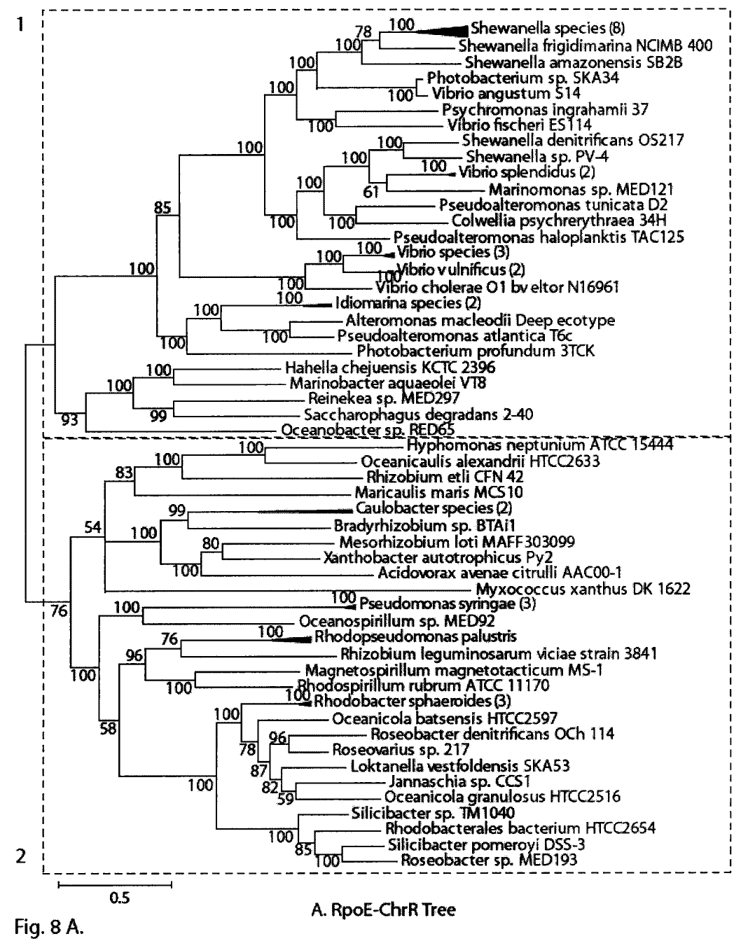

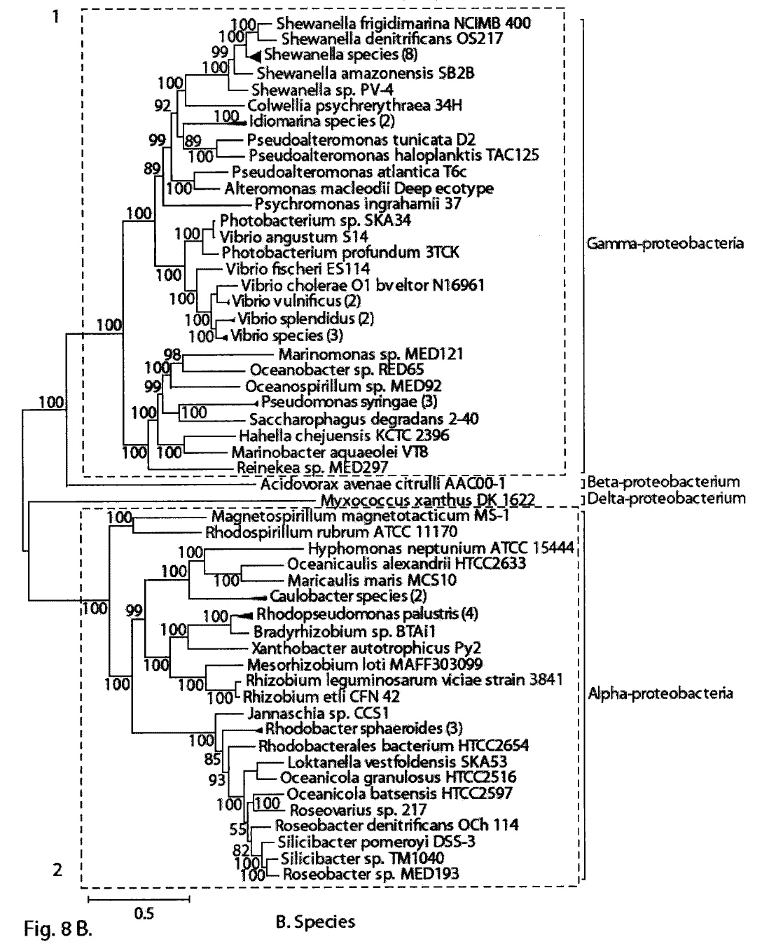

As evident from FIG. 8, the σE-ChrR tree generally mirrors the species tree. The only exceptions are Pseudomonas syringae species and Oceanospirillum sp. MED 92, members of the γ-proteobacteria, that contain σE-ChrR proteins that appear more related to those found in α-proteobacteria. In addition, the σE-ChrR proteins of β-proteobacteria and β-proteobacteria cluster with those found in the α-proteobacteria, suggesting lateral transfer of these genes into these species. Taken together, this analysis suggests that the σ^{E}-ChrR pair evolved before the branching of the α-proteobacteria and γ-proteobacteria.

To characterize the putative σ^{E}-ChrR regulon across these bacteria, the inventors analyzed the 73 bacterial genomes for possible orthologs of genes within the R. sphaeroides σ^{E}-ChrR regulon and genes predicted to contain σ^{E} promoters. Some of these 73 genomes were not completely assembled, so not all potential σ^{E} target genes might be captured. A two-step process was used to predict members of the σ^{E} regulon in these bacteria based on the R. sphaeroides σ^{E} promoter motif and the known target genes.

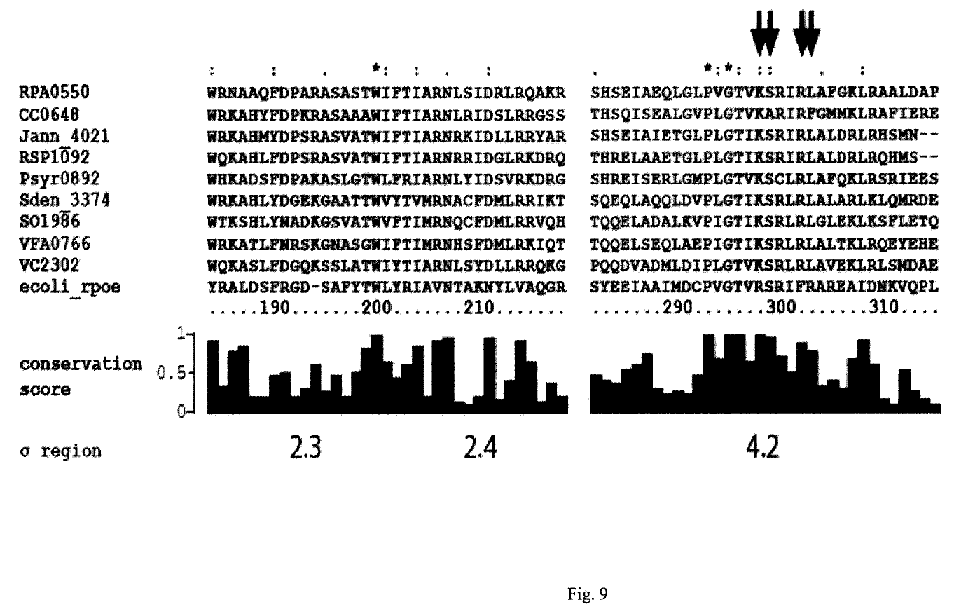

The first step was to identify candidate promoters in these 73 genomes. This prediction was based on amino acid conservation across the 73 R. sphaeroides σ^{E} homologs. The inventors predicted the residues of the 73 R. sphaeroides σ^{E} homologs that are involved in the −35 promoter element recognition based on the known Escherichia coli σ^{E} residues that contact the −35 promoter element. Because these residues were conserved among the 73 R. sphaeroides σ^{E} homologs, the inventors hypothesized that the sequence of the −35 promoter element recognized by these proteins is conserved. While information about interactions between domains 2.3-2.4 and the −10 promoter element was not available for group IV σ factors, alignments of the 73 homologs revealed a high degree of amino acid sequence conservation in this region (FIG. 9), suggesting that the R. sphaeroides σ^{E} promoter motif could be used to query for potential target genes in the 73 bacterial genomes that contain σ^{E}-ChrR homologs.

To identify σ^{E}-ChrR regulon orthologs, the inventors performed a de novo search for the R. sphaeroides σ^{E} promoter motif upstream of rpoEchrR in other bacteria. This query identified a sequence upstream of a putative σ^{E} structural gene in 57 of these 73 microbes that is almost identical with that of the R. sphaeroides σ^{E} motif (FIG. 5B). A phylogenetically determined PSWM, constructed using the putative promoter sequences upstream of these 57 rpoEchrR operons, was almost identical with that of the R. sphaeroides σ^{E} motif. The phylogenetically determined σ^{E} PSWM was used to score all upstream regions in these 73 genomes to identify candidate members of the σ^{E}-ChrR regulons. To increase the sensitivity of the analysis, the threshold used to score positive matches was set low, which resulted in an increased number of possible false positives. However, these presumed false-positive matches were not conserved across the phylogeny.

To determine if groups of orthologous genes, constructed using OrthoMCL, were consistently associated with a predicted σ^{E} promoter, orthologs identified downstream of a candidate σ^{E} promoter were scored to reflect their potential membership in the σ^{E}-ChrR regulon. To account for the existence of σ^{E}-dependent operons, genes likely to be cotranscribed with one containing a match to the σ^{E} promoter motif were considered positive. After each protein coding sequence had been scored, ortholog group-specific values were calculated and ranked by summing individual values within each group.

The dataset of ranked orthologous proteins was divided into proteins found in α-proteobacteria or γ-proteobacteria (data derived from one δ-proteobacterium and one β-proteobacterium were removed, since they did not exhibit any informative pattern). The σ^{E} (protein ID 21307) and ChrR (protein ID 20362) homologs ranked highest in both α-proteobacteria and γ-proteobacteria as expected, since their expression should be positively autoregulated (see above). The remaining highly ranked groups from both α-proteobacteria and γ-proteobacteria contain orthologs of known members of the R. sphaeroides σ^{E}-ChrR regulon (RSP1087; protein ID no. 20348; RSP1088, protein ID no. 21508; RSP1090, protein ID no. 20013; RSP1091, protein ID no. 21520; RSP2143, protein ID no. 21876; RSP2144, protein ID no. 254; Table 9 (manuscript 3)). The presence of a σ^{E} promoter motif upstream of these genes supports their assignment to a core set of genes for this regulon and suggests that some part of the biological response controlled by σ^{E} and ChrR is conserved in α-proteobacteria and γ-proteobacteria. Except for RSP2143 and RSP2144 (DNA photolyase and cyclopropane fatty acid synthase, respectively), the biological functions of most of these conserved proteins are unknown.

TABLE 9
Functional annotation of selected groups of homologous
genes across the species considered in this study.

| | | | |
|---|---|---|---|
| Ortho | Rsph | Rsph gene | |
| ID | locus | name | Annotation |

The conserved core σ^{E}-ChrR regulon

| | | | |
|---|---|---|---|
| 254 | RSP2144 | cfaS | Cyclopropane-fatty acyl-phospholipid synthase |
| 20013 | RSP1090 | | Uncharacterized conserved protein |
| 20348 | RSP1087 | | Putative short-chain dehydrogenase/reductase |
| 20362 | RSP1093 | chrR | Anti-σ factor, anti-RpoE |
| 21307 | RSP1092 | rpoE | Alternative σ factor, group IV |
| 21508 | RSP1088 | | Uncharacterized protein |
| 21520 | RSP1091 | | Putative protein binding nicotinamide adenine |
| | | | dinucleotide/flavin adenine dinucleotide |
| 21876 | RSP2143 | phrB | Deoxyribodipyrimidine photolyase |

The extended σ^{E}-ChrR regulon
In R. sphaeroides

| | | | |
|---|---|---|---|
| 2073 | RSP1089 | | Putative Na⁺/melibiose symporter |
| 19916 | RSP1409 | | Protein containing fasciclin-like repeats |
| 20947 | RSP0601 | rpoH_{II} | Alternative σ factor from the σ³² family |
| 21322 | RSP1852 | | Uncharacterized conserved protein |
| 21508 | RSP1088 | | Uncharacterized protein |
| 21842 | RSP0296 | cycA | Cytochrome c₂ |
| 22229 | RSP3336 | | ABC transporter, inner membrane subunit |
| 22678 | RSP6222 | | Uncharacterized protein |

In Shewanella or Vibrio species

| | | | |
|---|---|---|---|
| 71 | RSP2389 | | Putative glutathione peroxidase |
| 127 | RSP2633 | ccmF | Cytochrome c maturation protein |
| 157 | RSP1805 | ccmG | Thiol-disulfide isomerase and thioredoxins |
| 791 | | | Uncharacterized protein |
| 884 | | | Putative sodium/glutamate symporter |
| 2176 | | | Uncharacterized conserved protein |
| 19769 | RSP3077 | | Putative deoxyribodipyrimidine photolyase |
| 20054 | RSP1803 | ccmC | ABC heme exporter, inner membrane subunit |
| 20172 | | | Putative acyl-CoA dehydrogenases |
| 21430 | | | Putative nucleoside diphosphate sugar epimerase |
| 21552 | | | Uncharacterized conserved protein |
| 22038 | RSP2945 | ccmE | Cytochrome-c-type biogenesis protein |
| 22231 | | | Putative small polyketide cyclase or steroid isomerase |
| 23701 | | | Adenosylmethionine-8-amino-7-oxononanoate aminotransferase |
| 2386 | RSP3424 | | Putative dehydrogenase |

The corresponding R. sphaeroides locus ID and gene names are indicated when existing in the respective groups.
J Mol Biol. Author manuscript; available in PMC 2008 Nov. 14.

Some ortholog groups appear to be specific to the α-proteobacteria or the γ-proteobacteria. For example, RSP0601 (rpoH_{II}, protein ID no. 20947), which encodes a second member of the σ³² family, is part of the R. sphaeroides σ^{E}-ChrR regulon. It is therefore not surprising to find rpoH_{II} orthologs that appear to contain a σ^{E} promoter in α-proteobacteria such as Roseovarius sp. 217, Hyphomonas neptunium ATCC 15444, Oceanicaulis alexandrii HTCC2633, Jannaschia sp. CCS1, Loktanella vestfoldensis SKA53, Maricaulis maris MCS10, and Silicibacter pomeroyi DSS3. However, other α-proteobacteria contain an rpoH_{II} gene that lacks elements related to the conserved σ^{E} promoter motif even though this sequence is present upstream of other predicted core members of this regulon. In addition, the predicted σ^{E}-ChrR regulons in these a-proteobacteria do not appear to be extended to compensate for the loss of any RpoH_{II} target gene, raising questions about the existence of a second tier of ¹O₂-responsive genes in these species and the exact role of RpoH_{II} in bacteria containing this gene product.

In the γ-proteobacteria that contain rpoEchrR operons, no rpoH_{II} orthologs were found. On the other hand, the motif search revealed that a putative polyketide cyclase specific for the γ-proteobacteria often possesses a σ^{E} binding motif in its promoter region. In addition, this gene is generally found in the genomic neighborhood of orthologs of the R. sphaeroides σ^{E}-regulated genes in these γ-proteobacteria.

Several species of α-proteobacteria and γ-proteobacteria do not have the conserved σ^{E} binding motif associated with any of the predicted conserved regulon members. To determine if the σ^{E} promoter motif evolved into a different sequence, the inventors queried the promoter regions of possible regulon members in each species individually for a conserved sequence that related to a σ factor binding site. No conserved motif with high information content could be found. It is possible that σ^{E} is not functional or that both the σ^{E} binding motif and the regulon composition have diverged in these bacteria. However, no evidence for such divergence was evident in sequence alignments of their σ^{E} homologs in regions 2.3-2.4 and 4.2.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

The invention claimed is:

1. A method for inhibiting growth of bacteria exposed to singlet oxygen, the method comprising the steps of:
reducing availability in the bacteria of sigma factor σ^{E} to a level insufficient to activate a σ^{E} regulon such that bacterial growth is inhibited.

2. A method as recited in claim 1, wherein the reducing step includes the step of providing in the bacteria a σ^{E}-binding anti-sigma agent selected from the group consisting of ChrR and a fragment of ChrR.

3. A method as recited in claim 2, wherein the fragment of ChrR comprises at least amino acids 1-85 of SEQ ID NO:1.

4. A method as recited in claim 2, wherein the fragment of ChrR consists of amino acids 1-85 of SEQ ID NO:1.

5. A method as recited in claim 1, wherein the bacteria are Rhodobacter sphaeroides.

6. A method as recited in claim 1, wherein the bacteria are a Vibrio species.

7. A method for protecting a cellular organism from damage in the presence of singlet oxygen, the method comprising the steps of:
reducing binding in cells of the organism between σ^{E} and a σ^{E}-binding anti-sigma agent selected from the group consisting of ChrR and a fragment of ChrR.

8. A method as recited in claim 6, wherein the reducing step comprises the step of introducing into a gene encoding σ^{E} (SEQ ID NO:2) a mutation selected from the group consisting of a K38E mutation, a K38R mutation and a M42A mutation.

9. A method as recited in claim 6, wherein the reducing step comprises the step of introducing into a gene that encodes the anti-sigma agent (SEQ ID NO:1) having a mutation selected from the group consisting of a H6A mutation, a H31A mutation, a C35A mutation, a C35S mutation, a C38A mutation, a C38S mutation, a C38R mutation and a C187/189S mutation.

10. A method as recited in claim 6, wherein the reducing step comprises the step of eliminating the anti-sigma agent from the cells.

11. A method as recited in claim 6, wherein the cellular organism is selected from the group consisting of a bacterium and an alga.

12. A method as recited in claim 6, wherein the organism produces a commodity chemical product.

13. A method as recited in claim 11, wherein the commodity product is selected from the group consisting of acetic acid, acetone, acrylamide, butanol, ethanol, glycerol, hydrogen peroxide and lactic acid.

14. A method as recited in claim 10, wherein the bacterium is Rhodobacter sphaeroides.

15. A method for preventing damage in a organism to membrane lipids exposed to singlet oxygen, the method comprising the step of:
increasing expression of a σ^{E}-responsive gene in the organism.

16. A method as recited in claim 14, wherein the organism is selected from the group consisting of bacteria, algae and plants.

17. A method as recited in claim 14, wherein the gene encodes for cyclopropane-fatty-acyl-phospholipid synthase in order to produce strategic compounds like hydrocarbons of protective value against singlet oxygen or of commercial value as a biofuel, lubricant or commodity chemical.

18. A method for inhibiting growth of bacteria exposed to singlet oxygen, the method comprising the step of:
reducing activity of a σ^{E} regulon such that bacterial growth is inhibited.

19. A method of claim 18, wherein the reducing step comprises altering a sigma factor σ^{E} promoter binding site (SEQ ID No. 10) such that sigma factor σ^{E} binding to the promoter is inhibited.

20. A method of claim 19, wherein the altering step of claim 19 includes the step of introducing a substitution in place of the first T in a −35 region of the promoter binding site.
